SEQUENCE LISTING (4 sequences)

SEQ ID NO: 1 - protein, 163 aa, Homo sapiens
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 2 - protein, 9 aa, Homo sapiens
YFPGQFAFS

SEQ ID NO: 3 - protein, 10 aa, synthetic construct
  SITE 1: note = D-Tyrosine
  SITE 5: note = D-Serine
  SITE 8: note = D-Alanine
  SITE 9: note = 1,2,3,4 tetrahydroisoquinoline-3-carboxylic acid
YFLPSQFAXS

SEQ ID NO: 4 - protein, 137 aa, Homo sapiens
ELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFS

(19) United States
(12) Patent Application Publication
(10) Pub. No.: US 2026/0183369 A1
(43) Pub. Date: Jul. 2, 2026

(54) CHEMERIN-CONTAINING FORMULATION FOR TREATING DRY EYE DISEASE

(71) Applicant: OKYO Pharma Limited, New York, NY (US)

(72) Inventor: Rajkumar V. PATIL, Keller, TX (US)

(21) Appl. No.: 18/864,704

(22) PCT Filed: May 11, 2023
(86) PCT No.: PCT/US2023/066879

§ 371 Date: Nov. 11, 2024

Related U.S. Application Data
(60) Provisional application No. 63/341,205, filed on May 12, 2022.
(60) Provisional application No. 63/434,042, filed on Dec. 20, 2022.

(51) Int. Cl. A61K 38/19; A61K 47/02; A61K 47/54; A61P 27/04

(52) U.S. Cl. CPC A61K 38/19; A61K 47/02; A61K 47/543; A61K 47/545; A61P 27/04

(57) ABSTRACT

The present disclosure relates to among other things, an ophthalmic formulation of a lipidated chemerin composition comprising (a) chemerin or a fragment or analog thereof and (b) a lipid entity linked to the chemerin or fragment or analog thereof, that is stable in terms of pH, osmolality, physical appearance, and purity of the chemerin composition, when stored under normal storage conditions. One aspect of the present disclosure also relates to use of the said ophthalmic formulation for treating an inflammatory condition including, but not limited to, ocular inflammation, dry eye disease, and ocular neuropathic pain.

### CROSS-REFERENCE TO RELATED APPLICATIONS

This application claims priority to U.S. Provisional Patent Application Nos. 63/341,205 filed on May 12, 2022, and 63/434,042 filed on Dec. 20, 2022, the contents of each of which are incorporated herein by reference in their entireties.

### INCORPORATION BY REFERENCE OF SEQUENCE LISTING

The contents of the electronic sequence listing (OKYO_008_001WO_SeqList_ST26.xml; Size: 6,273 bytes; and Date of Creation: Apr. 19, 2023) are herein incorporated by reference in their entirety.

### BACKGROUND OF THE DISCLOSURE

There are a variety of inflammatory conditions that affect the eye, including, but not limited to, ocular inflammation, dry eye disease (DED), and ocular neuropathic pain. Ocular inflammation can be caused by a microbial infection of the eye. Such infection may be fungal, viral, or bacterial. Ocular inflammation can also be caused by trauma, burn, autoimmune disease, chemical injury, contact lens, or other external stimuli. Neuropathic pain is a major health problem that occurs in as much as 7% of the general population. Up to 50% of patients do not respond to standard therapy.

DED is a multifactorial disease of the tears and the ocular surface with inflammation playing a part in its pathogenesis. Dry eye is a common and often chronic problem, particularly in older adults. In 2000, its prevalence in the US has been estimated around 17% in females and 12% in males but it has been increased in recent years and estimated to be more than 50%. People with dry eyes either do not produce enough tears or their tears are of a poor quality. Tears are produced by several glands in and around the eyelids. Tear production tends to diminish with age, with various medical conditions or as a side effect of certain medicines. Environmental conditions, such as wind and dry climates, can also decrease tear volume due to increased tear evaporation. When the normal amount of tear production decreases or tears evaporate too quickly from the eyes, symptoms of dry eye can develop. As for the quality of tears, tears are made up of three layers: oil, water and mucus. Each component protects and nourishes the front surface of the eye. A smooth oil layer helps prevent evaporation of the water layer, while the mucin layer spreads the tears evenly over the surface of the eye. If the tears evaporate too quickly or do not spread evenly over the cornea due to deficiencies with any of the three tear layers, dry eye symptoms can develop. The common form of dry eyes occurs when the water layer of tears is inadequate. This condition is also called keratoconjunctivitis sicca (KCS).

Current therapies for dry eye are only palliative, focusing on replacement of tears to reduce symptoms. Five approved products are available in the United States are: Restasis®, Xiidra®, CEQUA™, Eysuvis™ and TYRVAYA™. Restasis® (cyclosporine ophthalmic emulsion, 0.05%) a calcineurin inhibitor immunosuppressant, is a topical immune-modulator with anti-inflammatory effects. Although approved for use in treatment of DED in the US, most of these products cause adverse side effects including ocular irritation, dysgeusia, instillation site pain and conjunctival hyperemia.

OK-101 is a lipidated chemerin peptide designed to bind with high affinity to ChemR23 receptors. Binding of OK-101 to ChemR23 has been shown to produce anti-inflammatory activity in mouse models of DED. OK-101 was developed using a membrane-anchored-peptide (MAP) technology to produce a novel long-acting drug candidate for treating DED. OK-101 is also designed to combat washout through the inclusion of a lipid ‘anchor’ within its molecular structure to enhance residence time of OK-101 within the ocular environment. OK-101 has also been shown to resolve the inflammation in animal models of asthma and modulate the inflammation environment in autoimmune diseases by recruiting regulatory T cells (Tregs), as well as attenuating neuropathic pain in mice. (Stevenson, Chauhan et al. 2012, Doyle, Krishnaji et al. 2014). OKYO's studies demonstrated significant reduction in corneal permeability with topical application of OK-101 (0.04%) vs vehicle in an experimental model of dry eye disease in mice. In addition, OK-101 normalized goblet cell density and reduced count of CD4+ T cells, (biomarkers of inflammation) and increased T regulatory cells in the draining lymph node of OK-101 treated mice compared to vehicle in in the dry eye mice model. In addition, in a separate set of animal model experiments, OK-101 was shown to exhibit potent ocular pain-reducing activity in a mouse model of corneal neuropathic pain. With potential anti-inflammatory and analgesic characteristics, OK-101 is currently developed by OKYO Pharma US, Inc. for the treatment of dry eye disease.

There is a need for a therapeutic formulation for treatment of ocular inflammatory conditions like DED, with fewer to no adverse effects and that is well tolerated by the patient's eye. For this the stability of such ophthalmic formulations in terms of variability in pH, osmolality etc., is important.

The present disclosure addresses this need of patients suffering from various inflammatory conditions including, but not limited to, ocular inflammation, DED, and ocular neuropathic pain, by providing an ophthalmic formulation of OK-101 that in composition is close to human tears and is stable in terms of pH, osmolality, physical appearance, and purity, over at least one to 6 months.

### SUMMARY OF THE DISCLOSURE

The present disclosure provides an ophthalmic formulation comprising: (a) about 0.1% to about 0.5% w/v of NaCl; (b) about 25 mM to about 100 mM of phosphate buffered saline; and (c) about 0.05% to about 0.1% w/v of a lipidated chemerin composition that includes a chemerin fragment consisting of the sequence of Y-F-P-G-Q-F-A-F-S (SEQ ID NO: 2) or a chemerin analog consisting of the sequence of Y*-F-L-P-S*-Q-F-A*-Tic-S (SEQ ID NO: 3), wherein * denotes D amino acids and Tic represents 1,2,3,4 tetrahydroisoquinoline-3-carboxylic acid, the chemerin fragment or chemerin analog being linked to a lipid entity via a linker; wherein the formulation has a pH of about 6.5 to about 8.5, and an osmolality of about 200 to about 450 mOsm/Kg.

The present disclosure also provides a method of treating an inflammatory condition in a subject in need thereof, the method comprising topically administering to an eye of the subject a therapeutically effective amount of the formulation disclosed herein.

The present disclosure also provides a method of treating pain in a subject in need thereof, the method comprising topically administering to an eye of the subject a therapeutically effective amount of the formulation disclosed herein.

The present disclosure also provides a kit for administration of the ophthalmic formulation disclosed herein to a subject in need thereof. Also provided are a method of making the ophthalmic formulation disclosed herein.

Any aspect or embodiment described herein can be combined with any other aspect or embodiment as disclosed herein. While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

### BRIEF DESCRIPTION OF THE DRAWINGS

FIGS. 1A-1B are charts showing change in pH and solubility of the lipidated chemerin composition of the present disclosure (OK-101) in 100 mM sodium phosphate buffer (FIG. 1A) and 50 mM sodium phosphate buffer (FIG. 1B). The pH values and concentration of OK-101 achieved are as indicated.

FIGS. 2A-2D show the accuracy and detection limits of the HPLC assay used for detecting OK-101. FIG. 2A shows the concentration of OK-101 (mg/ml) plotted against the detection signal peak area. FIG. 2B shows the detection signal peak area for the repeat injections (input) of the first standard of FIG. 2A, to show consistency and accuracy of detection. FIG. 2C is the standard curve representative of FIG. 2A. FIG. 2D shows the different 0.2 μm membranes tested and considered for use in the HPLC assay.

FIG. 3 is a chart showing the conditions and schedule of testing the formulations containing 0.05% OK-101 product in a 100 mM sodium phosphate buffer with 0.3% NaCl and 0.05% OK-101 product in a 100 mM sodium phosphate buffer with 1.4% mannitol. Specific testing are indicated as follows: A-Testing to be performed for A include appearance, pH, osmolality, color, assay/potency, and impurities by HPLC; and S-Storage only.

FIG. 4A-4E show change in appearance, pH, osmolality, purity of formulations of OK-101 product over a storage period of one month. FIG. 4A is a picture showing the clear and colorless appearance of the formulation of 0.05% OK-101 product in a 100 mM sodium phosphate buffer with 0.3% NaCl and the formulation of 0.05% OK-101 product in a 100 mM sodium phosphate buffer with 1.4% mannitol, stored under 40° C./75% RH and 25° C./60% RH storage conditions. FIG. 4B is a chart showing the change in pH, osmolality, % assay purity and the % area values for each formulation and storage condition corresponding to each related retention time (RRT), for T-0 and T=1 month, for each formulation and storage condition are as indicated. FIGS. 4C, 4D and 4E are charts showing the purity of the OK-101 product and the formulation as determined using HPLC assay, at T=0, T=1 month under 40° C./75% RH storage condition and T=1 month under 25° C./60% RH. The % assay purity levels and the % area values for each formulation and storage condition corresponding to each related retention time (RRT) are as indicated.

FIG. 5A-5C show change in appearance, pH, osmolality, purity of formulations of 0.05% w/v OK-101 product over a storage period of two months, three months, and 6 months. FIG. 5A shows the change in appearance, pH, osmolality, purity of formulations of 0.05% OK-101 product in a 100 mM sodium phosphate buffer with 0.3% NaCl and the formulation of 0.05% OK-101 product in a 100 mM sodium phosphate buffer with 1.4% mannitol, stored for T=2 and T=3 months, under 40° C./75% RH and 25° C./60% RH storage conditions. The pH values, osmolality values (mOsm), % assay purity levels and the % area values for each formulation and storage condition corresponding to each related retention time (RRT), for each formulation and storage condition are as indicated. FIG. 5B is a chart showing the change in appearance, pH, osmolality, % assay purity levels of formulation of 0.05% OK-101 product in a 100 mM sodium phosphate buffer with 0.3% NaCl, and the formulation of 0.05% OK-101 product in a 100 mM sodium phosphate buffer with 1.4% mannitol, stored for T=6 months, under 40° C./75% RH and 25° C./60% RH storage conditions. FIG. 5C is a picture showing the clear and colorless appearance of the formulation of 0.05% OK-101 product in a 100 mM sodium phosphate buffer with 0.3% NaCl, and the formulation of 0.05% OK-101 product in a 100 mM sodium phosphate buffer with 1.4% mannitol, stored under 40° C./75% RH and 25° C./60% RH storage conditions, for a period of 3 months.

FIG. 6A-6B show change in appearance, pH, osmolality, purity of formulations of 0.1% w/v of OK-101 product over a storage period of one month and two months. FIG. 6A is a chart showing the conditions and schedule of testing the formulations containing 0.05% OK-101 product in a 100 mM sodium phosphate buffer with 0.3% NaCl and 0.05% OK-101 product in a 100 mM sodium phosphate buffer with 1.4% mannitol. Specific testing are indicated as follows: A-Testing to be performed for A include appearance, pH, osmolality, color, assay/potency, and impurities by HPLC; and S-Storage only. FIG. 6B is a chart showing the change in appearance, pH, osmolality, % assay purity levels of formulation of 0.05% OK-101 product in a 100 mM sodium phosphate buffer with 0.3% NaCl, and the formulation of 0.05% OK-101 product in a 100 mM sodium phosphate buffer with 1.4% mannitol, stored for T=1 months and T-2 months, under 40° C./75% RH and 25° C./60% RH storage conditions. The pH values, osmolality values (mOsm) and % assay purity levels for each formulation and storage condition, are as indicated.

### DETAILED DESCRIPTION OF THE DISCLOSURE

The present disclosure provides an ophthalmic formulation comprising: (a) about 0.1% to about 0.5% w/v of NaCl; (b) about 25 mM to about 100 mM of phosphate buffered saline; and (c) about 0.05% to about 0.1% w/v of a lipidated chemerin composition that includes a chemerin fragment consisting of the sequence of Y-F-P-G-Q-F-A-F-S (SEQ ID NO: 2) or a chemerin analog consisting of the sequence of Y*-F-L-P-S*-Q-F-A*-Tic-S (SEQ ID NO: 3), wherein * denotes D amino acids and Tic represents 1,2,3,4 tetrahydroisoquinoline-3-carboxylic acid, the chemerin fragment or chemerin analog being linked to a lipid entity via a linker; wherein the formulation has a pH of about 6.5 to about 8.5, and an osmolality of about 200 to about 450 mOsm/Kg.

In some embodiments, the formulation of the present disclosure comprises 0.1% to 0.5% w/v of NaCl (e.g., 0.1% to 0.2%, 0.2% to 0.3%, 0.3% to 0.4% or 0.4% to 0.5% w/v of NaCl). In some embodiments, the formulation of the present disclosure comprises 0.1% to 0.3% w/v of NaCl (e.g., 0.1% to 0.2% or 0.2% to 0.3% w/v of NaCl). In some embodiments, the formulation of the present disclosure comprises 0.3% w/v of NaCl.

In some embodiments, the formulation of the present disclosure comprises 0.05% to 0.1% w/v (e.g., 0.05% to 0.075%, 0.075% to 0.1% w/v) of a lipidated chemerin composition disclosed herein. In some embodiments, the formulation of the present disclosure comprises about 0.05% of a lipidated chemerin composition disclosed herein. In some embodiments, the formulation of the present disclosure comprises 0.05% of a lipidated chemerin composition disclosed herein. In some embodiments, the formulation of the present disclosure comprises about 0.1% of a lipidated chemerin composition disclosed herein. In some embodiments, the formulation of the present disclosure comprises 0.1% of a lipidated chemerin composition disclosed herein.

In some embodiments, the formulation of the present disclosure comprises 25 mM to 50 mM (e.g., 25 mM to 30 mM, 30 mM to 35 mM, 35 mM to 40 mM, 40 mM to 45 mM or 45 mM to 50 mM,) phosphate buffered saline. In some embodiments, the formulation of the present disclosure comprises 50 mM to 100 mM (e.g., 50 mM to 55 mM, 55 mM to 60 mM, 60 mM to 65 mM, 65 mM to 70 mM, 70 mM to 75 mM, 75 mM to 80 mM, 80 mM to 85 mM, 85 mM to 90 mM, 90 mM to 95 mM or 95 mM to 100 mM) phosphate buffered saline. In some embodiments, the formulation of the present disclosure comprises 50 mM phosphate buffered saline. In some embodiments, the formulation of the present disclosure comprises 100 mM phosphate buffered saline.

In some embodiments, the formulation of the present disclosure has a pH of 6.5 to 8.5 (e.g., 6.5 to 7.0, 7.0 to 7.5, 7.5 to 8.0 or 8.0 to 8.5). In some embodiments, the formulation of the present disclosure has a pH of about 7.2 to about 7.4. In some embodiments, the formulation of the present disclosure has a pH of 7.3 to 7.4. In some embodiments, the formulation of the present disclosure has a pH of 7.4 to 7.6.

In some embodiments, the formulation of the present disclosure comprises an osmolality of 200 to 450 mOsm/Kg (e.g., 200 to 250, 250 to 300, 300 to 350, 350 to 400 or 400 to 450 mOsm/kg). In some embodiments, the formulation has an osmolality of about 290 to about 320 mOsm/kg (e.g., 290 to 300, 300 to 310, 310 to 315, 315 to 320, 320 to 325, 325 to 330, 330 to 335, 335 to 340, 340 to 345 or 345 to 350 mOsm/Kg). In some embodiments, the formulation of the present disclosure comprises an osmolality of 290 to 325 mOsm/Kg (e.g., 290 to 295, 295 to 300, 300 to 305, 305 to 310, 310 to 315, 315 to 325 mOsm/Kg). In some embodiments, the formulation of the present disclosure comprises an osmolality of 313 to 322 mOsm/Kg (e.g., 313 to 315, 315 to 317, 317 to 319 or 319 to 322 mOsm/Kg). In some embodiments, the formulation of the present disclosure comprises an osmolality of 314 to 319 mOsm/Kg (e.g., 314 to 315, 315 to 316, 316 to 317 or 318 to 319 mOsm/Kg).

In some embodiments, the pH of the formulation of the present disclosure change by no more than 1.5% (0% to 0.25%, 0.25% to 0.5%, 0.5% to 0.75%, 0.75% to 1%, 1% to 1.25% or 1.25% to 1.5%) over a period of one month to 6 month (e.g., one month to two months, two months to three months, three months to four months, four months to five months or five months to six months) when stored at 25° C. to 40° C. and 60% to 75% relative humidity.

In some embodiments, the pH of the formulation of the present disclosure change by 1% to 1.5% over a period of one month when stored at 25° C. and 60% relative humidity. In some embodiments, the pH of the formulation of the present disclosure change by 1% to 1.5% over a period of two months when stored at 25° C. and 60% relative humidity. In some embodiments, the pH of the formulation of the present disclosure change by 1% to 1.5% over a period of three months when stored at 25° C. and 60% relative humidity. In some embodiments, the pH of the formulation of the present disclosure change by 1% to 1.5% over a period of six months when stored at 25° C. and 60% relative humidity.

In some embodiments, the pH of the formulation of the present disclosure does not change over a period of one month when stored at 25° C. and 60% relative humidity. In some embodiments, the pH of the formulation of the present disclosure does not change over a period of two month when stored at 25° C. and 60% relative humidity. In some embodiments, the pH of the formulation of the present disclosure does not change over a period of three month when stored at 25° C. and 60% relative humidity. In some embodiments, the pH of the formulation of the present disclosure does not change over a period of six month when stored at 25° C. and 60% relative humidity.

In some embodiments, the pH of the formulation of the present disclosure change by 1% to 1.5% over a period of one month when stored at 40° C. and 75% relative humidity. In some embodiments, the pH of the formulation of the present disclosure change by 1% to 1.5% over a period of two months when stored at 40° C. and 75% relative humidity. In some embodiments, the pH of the formulation of the present disclosure change by 1% to 1.5% over a period of three months when stored at 40° C. and 75% relative humidity. In some embodiments, the pH of the formulation of the present disclosure change by 1% to 1.5% over a period of six months when stored at 40° C. and 75% relative humidity.

In some embodiments, the pH of the formulation of the present disclosure does not change over a period of one month when stored at 40° C. and 75% relative humidity. In some embodiments, the pH of the formulation of the present disclosure does not change over a period of two month when stored at 40° C. and 75% relative humidity. In some embodiments, the pH of the formulation of the present disclosure does not change over a period of three month when stored at 40° C. and 75% relative humidity. In some embodiments, the pH of the formulation of the present disclosure does not change over a period of six month when stored at 40° C. and 75% relative humidity.

In some embodiments, the osmolality of the formulation changes by no more than about 8% over a period of one months to six months when stored at 25° C. to 40° C. relative humidity. In some embodiments, the osmolality of the formulation changes by 0.3% to 10% (e.g., 0.3% to 0.5%, 0.5% to 1.0%, 1.0% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5% or 9.5% to 10%) over a period of one months to six months (e.g., one month to two months, two months to three months, three months to four months, four months to five months or five months to six months) when stored at 25° C. to 40° C. and 60% to 75% relative humidity.

In some embodiments, the osmolality of the formulation changes by no more than 2% (e.g., 0.1% to 0.25%, 0.25% to 0.3%, 0.3% to 0.5%, 0.5% to 0.75%, 0.75% to 1.0%, 1.0% to 1.25% or 1.25% to 1.5%) over a period of one months to six months (e.g., when stored at 25° C. and 60% relative humidity. In some embodiments, the osmolality of the formulation changes by 0.25% to 2% (e.g., 0.25% to 0.3%, 0.3% to 0.5%, 0.5% to 0.75%, 0.75% to 1.0%, 1.0% to 1.25% or 1.25% to 1.5%, 1.5% to 2%) over a period of one months to six months (e.g., when stored at 25° C. and 60% relative humidity. In some embodiments, the osmolality of the formulation changes by 0.25% to 2% over a period of one month when stored at 25° C. and 60% relative humidity. In some embodiments, the osmolality of the formulation changes by no more than 0.25% to 2% over a period of two months when stored at 25° C. and 60% relative humidity. In some embodiments, the osmolality of the formulation changes by 0.25% to 2% over a period of three months when stored at 25° C. and 60% relative humidity. In some embodiments, the osmolality of the formulation changes by 0.25% to 2% over a period of six months when stored at 25° C. and 60% relative humidity.

In some embodiments, the osmolality of the formulation of the present disclosure does not change over a period of one month when stored at 25° C. and 60% relative humidity. In some embodiments, the osmolality of the formulation of the present disclosure does not change over a period of two month when stored at 25° C. and 60% relative humidity. In some embodiments, the osmolality of the formulation of the present disclosure does not change over a period of three month when stored at 25° C. and 60% relative humidity. In some embodiments, the osmolality of the formulation of the present disclosure does not change over a period of six month when stored at 25° C. and 60% relative humidity.

In some embodiments, the osmolality of the formulation changes by no more than 8% (e.g., 1% to 1.5%, 1.5% to 2%, 2% to 2.5%, 2.5% to 5% or 5% to 5.5%, 5.5% to 6%, 6% to 6.5%, 6.5% to 7%, 7% to 7.5% or 7.5% to 8%) over a period of one months to six months (e.g., one month to two months, two months to three months, three months to four months, four months to five months or five months to six months) when stored at 40° C. and 75% relative humidity. In some embodiments, the osmolality of the formulation changes by 1% to 8% (e.g., 1% to 1.5%, 1.5% to 2%, 2% to 2.5%, 2.5% to 5% or 5% to 5.5%, 5.5% to 6%, 6% to 6.5%, 6.5% to 7%, 7% to 7.5% or 7.5% to 8%) over a period of one months to six months (e.g., one month to two months, two months to three months, three months to four months, four months to five months or five months to six months) when stored at 40° C. and 75% relative humidity. In some embodiments, the osmolality of the formulation changes by 1% to 8% to over a period of one month when stored at 40° C. and 75% relative humidity. In some embodiments, the osmolality of the formulation changes by 1% to 8% over a period of two month when stored at 40° C. and 75% relative humidity. In some embodiments, the osmolality of the formulation changes by 1% to 8% over a period of three month when stored at 40° C. and 75% relative humidity. In some embodiments, the osmolality of the formulation changes by 1% to 8% over a period of six month when stored at 40° C. and 75% relative humidity.

In some embodiments, the osmolality of the formulation of the present disclosure does not change over a period of one month when stored at 40° C. and 75% relative humidity. In some embodiments, the osmolality of the formulation of the present disclosure does not change over a period of two month when stored at 40° C. and 75% relative humidity. In some embodiments, the osmolality of the formulation of the present disclosure does not change over a period of three month when stored at 40° C. and 75% relative humidity. In some embodiments, the osmolality of the formulation of the present disclosure does not change over a period of six month when stored at 40° C. and 75% relative humidity.

In some embodiments, the purity of the lipidated chemerin composition used for making the formulation determined by HPLC is >94.6% (e.g., 94.6% to 95%, 95% to 96%, 96% to 97%, 97% to 98%, 98% to 99% or 99% to 100%) and the peptide content is >95.9% (e.g., 95.9% to 97%, 97% to 98% or 98% to 99% or 99% to 100%). In some embodiments, the purity of the lipidated chemerin composition used for making the formulation is 95% to 98% (e.g., 95% to 96%, 96% to 97% or 97% to 98%).

In some embodiments, in the lipidated chemerin composition used for making the formulation of the present disclosure, the Tic represents:

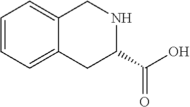

In some embodiments, in the lipidated chemerin composition used for making the formulation of the present disclosure, any of a variety of lipid entities may be utilized in accordance with the present disclosure. According to various embodiments, a lipid entity can comprise an entity capable of insertion into a lipid bilayer (e.g., a cell membrane). In some embodiments, a lipid entity is capable of incorporating into a lipid raft in a lipid bilayer (e.g., a cell membrane).

In some embodiments, the lipid entity can comprise a saturated or unsaturated fatty acid. The numbers in the lipid name are used to describe the fatty acid chains on the lipid. The numbers are generally presented in the format (number of carbons in fatty acid chain): (number of double bonds in fatty acid chain), e.g., 16:0 would be 16 carbons in the fatty acid chain with zero double bonds. The saturated or unsaturated fatty acid can include at least 4 carbons, at least 5 carbons, at least 6 carbons, at least 7 carbons, at least 8 carbons, at least 9 carbons, at least 10 carbons, or at least 15 carbons in the fatty acid chain. In some embodiments, the saturated or unsaturated fatty acid can include about 4-24 carbons in the fatty acid chain. The number of double bonds in the fatty acid chain can be in the range of 0-10, e.g., 0-8, 0-6, 1-8, 1-6. For example, the lipid entity can be C22:0, C22:1, C22:2, C22:3, C22:4, C22:5, C22:6, C20:0, C20:1, C20:2, C20:3, C20:4, C20:5, C20:6, C18:0, C18:1, C18:2, C18:3, C18:4, C18:5, C18:6, C10:0, C10:1, C10:2, C10:3, C10:4, etc.

For example, the lipid entity can be selected from the group consisting of α-linolenic acid, γ-linolenic acid, stearidonic acid, eicosapentaenoic acid, docosahexaenoic acid, linoleic acid, dihomo-γ-linolenic acid, arachidonic acid, docosatetraenoic acid, palmitoleic acid, vaccenic acid, paullinic acid, oleic acid, elaidic acid, gondoic acid, erucic acid, nervonic acid, mead acid, myristic acid, palmitic acid, stearic acid, 1,2-dipalmitoyl-sn-glycero-3-phosphoethanolamine (DPPE), GM1 ganglioside, GM2 ganglioside, GM3 ganglioside, 1,2-dipalmitoyl-sn-glycero-3-phosphocholine (DPPC), 1,2-dioleoyl-sn-glycero-3-phospho-L-serine (DOPS), 1,2-dioleoyl-sn-glycero-3-phosphocholine (DOPC), a glycosphingolipid, a sphingolipid, phosphatidylinositol 4,5-bisphosphate (PIP2), a ceramide, cholesterol, ergosterol, phytosterol, a hopanoid, a steroid, fluorinated-GM1, fluorinated-GM2, and fluorinated-GM3. In some embodiments, the lipid entity can be α-linolenic acid. In some embodiments, the lipid entity can be γ-linolenic acid. In some embodiments, the lipid entity can be palmitic acid. In some embodiments, the lipid entity can be vaccenic acid. In some embodiments, the lipid entity can be oleic acid. In some embodiments, the lipid entity can be elaidic acid.

The attachment of a lipid entity to a polypeptide is referred to as lipidation. In some embodiments, lipidation may comprise N-myristoylation. As used herein, “N-myristoylation” refers to the attachment of a myristate to an N-terminal glycine.

In some embodiments, lipidation may comprise palmitoylation. As used herein “palmitoylation” refers to the creation of a thioester linkage of long-chain fatty acids on one or more cysteine residues present in a peptide or protein.

In some embodiments, lipidation comprises GPI-anchor addition. As used herein “GPI-anchor addition” refers to the linkage of glycosyl-phosphatidylinositol (GPI) to the C-terminus of a protein.

In some embodiments, lipidation comprises prenylation. As used herein “prenylation” refers to the creation of a thioether linkage of an isoprenoid lipid (e.g., farnesyl (C-15) or geranylgeranyl (C-20)) to a cysteine present in a peptide or protein. In some embodiments, lipidation comprises geranylation. In some embodiments, lipidation includes geranylgeranylation. In some embodiments, lipidation comprises the association of a ligand entity with any compound that is soluble in a cellular membrane (e.g., 10:1 in equilibrium constant Kassoc≥10).

In some embodiments, lipidation may comprise one or more of the following: attachment of diacylglycerol to the side chain of an N-terminal cysteine of a peptide or protein via the sulfur atom; attachment of O-octanoyl to a serine or threonine of a peptide or protein; and attachment of S-archaeol to a cysteine of a peptide or protein. In some embodiments, lipidation may occur, for example, at any lysine, glutamic acid, aspartic acid, serine, threonine, cysteine, and/or tyrosine. In some embodiments where a chemerin analog comprises one or more ornithine, lipidation may occur at any ornithine.

In some embodiments, the lipid entity can be linked at or near the N-terminus of chemerin or fragment or analog thereof. In some embodiments, the lipid entity can be linked at or near the C-terminus of chemerin or fragment or analog thereof.

In some embodiments, lipidation may include fluorination. Fluorination can include the addition of one or more C6F13 chains. Without wishing to be bound by theory, it is thought that the presence of one or more C6F13 chains may allow a lipid entity to segregate from hydrocarbon lipid membrane components (see J. Am. Chem. Soc. 2007, 129, 9037-9043; J. Phsy. Chem. B, 2008, 112, 8250-8256; J. Am. Chem. Soc., 2009, 131, 12091-12093).

In some embodiments, the presence of at least one alkene in the structure of a lipid entity provides increased fluidity in a membrane (i.e., greater ability to move within the membrane) as compared to similar lipid entities lacking at least one alkene. In some embodiments, a lipid entity with greater fluidity is able to provide enhanced activity towards targets (e.g., receptors, ion channels, or enzymes) with a low density in a membrane. Without wishing to be bound by theory, it is possible that lipid entities with increased ability to move within a membrane are able to encounter a low-density target faster than a lipid entity with less mobility within a membrane.

In some embodiments, the lipidated chemerin composition used for making the formulation of the present disclosure, can optionally comprise a linker that links the lipid entity to chemerin or the fragment or analog thereof. For example, the linker can have a length of between about 2 Å and 175 Å, inclusive. In some embodiments, a linker is between 30 Å and 150 Å, inclusive.

In some embodiments, the linker can comprise a peptide. In some embodiments, a peptide linker is between about 2 and 20 amino acid residues in length. In some embodiments, a peptide linker is between about 5 and 10 amino acid residues in length. According to various embodiments, peptide linkers can be designed such that one or more a-helices are formed between chemerin or a fragment or analog thereof and a lipid entity. In some embodiments, a peptide linker may comprise a plurality of α-helices. In some embodiments, the plurality of α-helices is consecutive. In some embodiments, a plurality of α-helices is 2, 3, 4, 5, 6, 7, 8, 9, 10, 15, 20, or more α-helices.

In some embodiments, a peptide linker can comprise repeating units, for example a plurality of repeating glycine-asparagine (GN) units. In some embodiments, a peptide linker can comprise an epitope tag (e.g., a c-Myc tag) or other marker to allow for identification and/or characterization of provided agents and their fate in vitro and/or in vivo.

In some embodiments, the linker can comprise a non-peptide entity. In some embodiments, non-peptide linkers may be a synthetic polymer. According to various embodiments, the synthetic polymer may be any of a variety of lengths. In some embodiments, a linker comprising a synthetic polymer comprises a monomeric unit of the polymer. In some embodiments, a linker comprising a synthetic polymer comprises two or more monomeric units of a synthetic polymer (e.g., 2, 3, 4, 5, 6, 7, 8, 9, 10, 20, 30, 40, 50, 100 or more monomeric units).

In some embodiments, a linker can comprise at least one molecule of polyethylene glycol (PEG). Specific, non-limiting examples of suitable polymeric linkers include linkers with one or more monomeric units according to one of the following formulas:

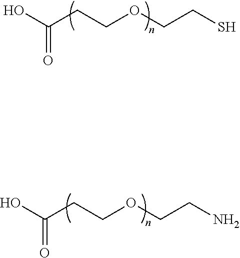

where n represents an integer greater than or equal to 1. In some embodiments, n is an integer between 2 and 50, 4 and 24, and/or 8 and 24, inclusive.

In some embodiments, in the lipidated chemerin composition used for making the formulation of the present disclosure, the linker is selected from the group consisting of:

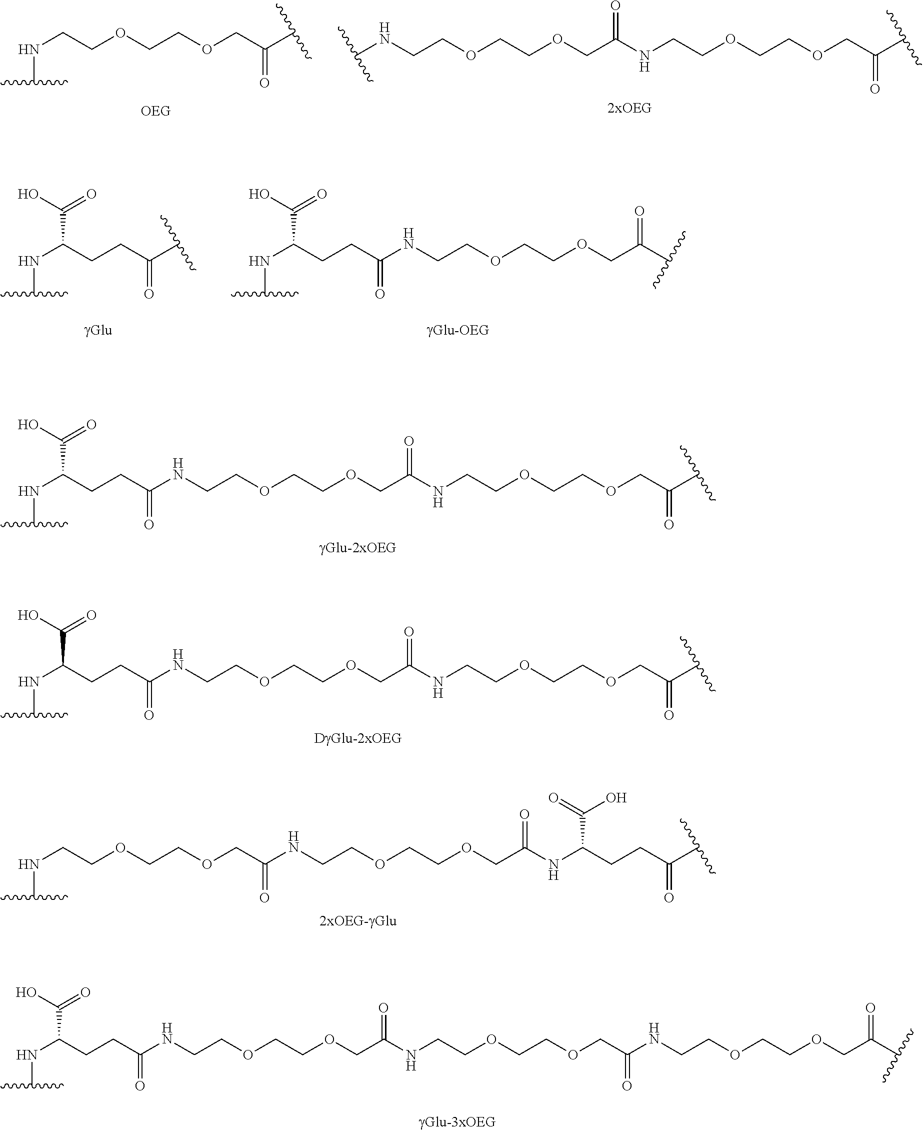

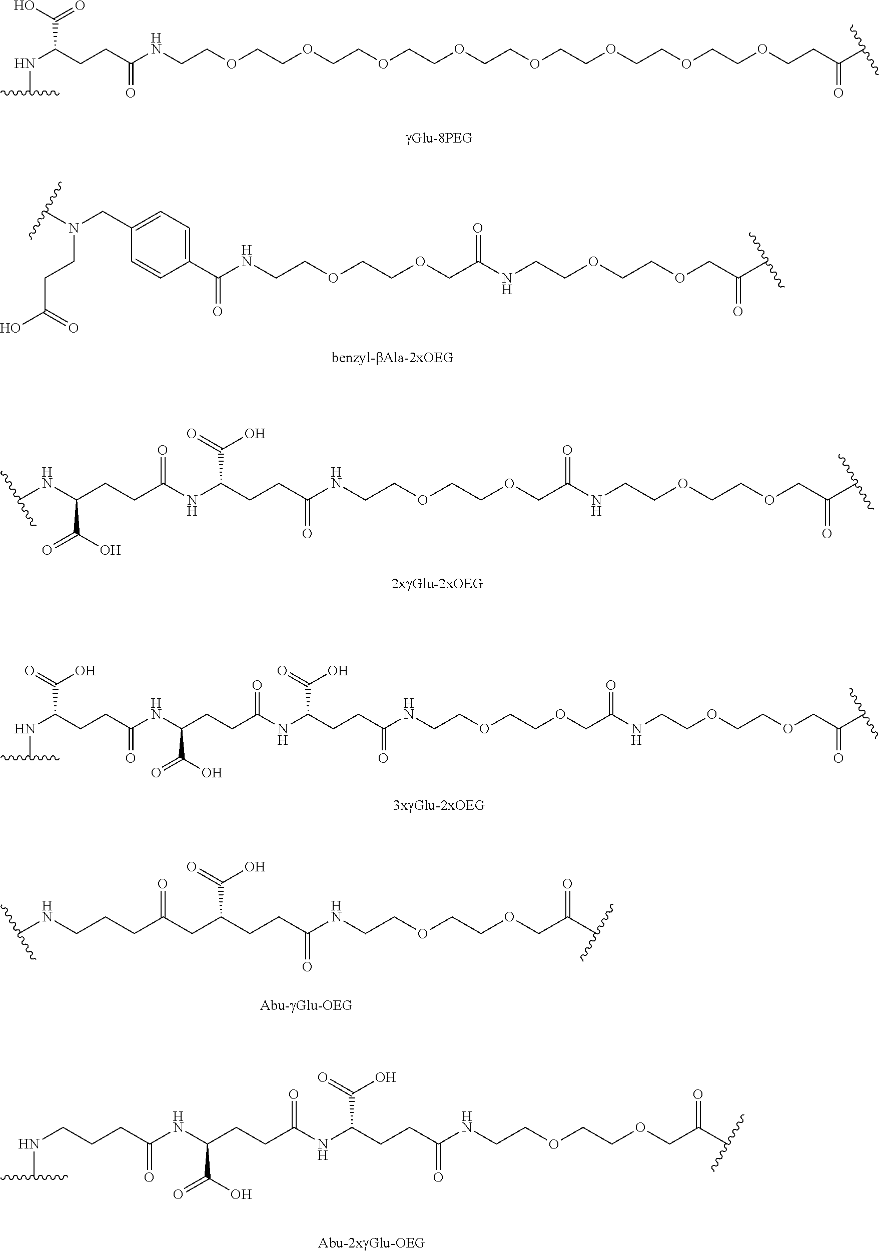 [SMILES: *N[C@@H](CCC(=O)NCCOCCOCCOCCOCCOCCOCCOCCOCCC(*)=O)C(=O)O ; *C(=O)COCCOCCNC(=O)COCCOCCNC(=O)c1ccc(CN(*)CCC(=O)O)cc1 ; *N[C@@H](CCC(=O)N[C@@H](CCC(=O)NCCOCCOCC(=O)NCCOCCOCC(*)=O)C(=O)O)C(=O)O ; *N[C@@H](CCC(=O)N[C@@H](CCC(=O)N[C@@H](CCC(=O)NCCOCCOCC(=O)NCCOCCOCC(*)=O)C(=O)O)C(=O)O)C(=O)O ; *NCCCC(=O)C[C@@H](CCC(=O)NCCOCCOCC(*)=O)C(=O)O ; *NCCCC(=O)N[C@@H](CCC(=O)N[C@@H](CCC(=O)NCCOCCOCC(*)=O)C(=O)O)C(=O)O]

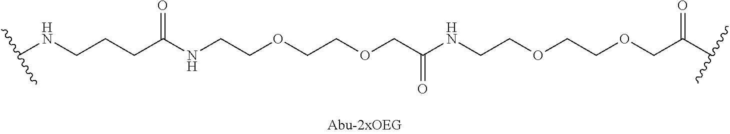 [SMILES: *NCCCC(=O)NCCOCCOCC(=O)NCCOCCOCC(*)=O]

In some embodiments, the linker comprises polyethylene glycol, GG, KGG, or a combination thereof.

In some embodiments, the lipid entity is linked at or near the N-terminus of the chemerin fragment or chemerin analog. In some embodiments, the lipid entity is linked at or near the C-terminus of the chemerin fragment or chemerin analog.

In some embodiments, a linker can comprise 1-Ethyl-3-(3-Dimethylaminopropyl) carbodiimide (EDAC), Benzophenone-4-Isothiocyanate, Bis-((N-Iodoacetyl) Piperazinyl) Sulfonerhodamine, Succinimidyl 2-(2-Pyridyldithio) Propionate (SPDP), 4-Azido-2,3,5,6-Tetrafluorobenzoic acid (ATFB), (N-((2-Pyridyldthio) ethyl)-4-Azidosalicylamide), Succinimidyl trans-4-(maleimidylmethyl)cyclohexane-1-carboxylate (SMCC), and/or N-(t-BOC)-aminooxyacetic acid. Those of skill in the art will be able to identify additional candidate linkers according to known methods.

In some embodiments, a linker can comprise both a peptide and a non-peptide entity.

In some embodiments, a linker is formed, at least in part, as a result of a click reaction as further described below. In some embodiments, the click reaction is an azide-alkyne Huisgen cycloaddition reaction.

Additional examples of lipid entities, linkers, and methods of lipidation can be found at US20160052982, the contents of which are incorporated herein by reference.

In some embodiments, the lipidated chemerin composition has the following structure:

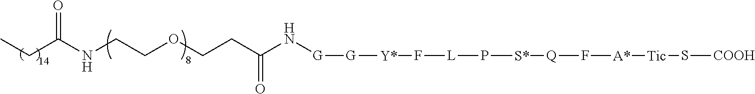

In some embodiments, the formulation of the present disclosure is formulated for topical administration as eye drops.

CMKLR1 is a G protein-coupled receptor which has been shown to modulate nociception. This receptor is expressed in glia, dorsal root ganglion neurons, and immune cells. The endogenous ligand (agonist) for CMKLR1 is chemerin, a 163 amino acid protein. Chemerin, also known as retinoic acid receptor responder protein 2 (RARRES2), tazarotene-induced gene 2 protein (TIG2), or RAR-responsive protein TIG2 is a protein that in humans is encoded by the RARRES2 gene. The amino acid sequence of chemerin (Homo sapiens) is shown below in SEQ ID NO: 1.

NCBI Reference Sequence: NP_002880.1

(SEQ ID NO: 1)
MRRLLIPLAL WLGAVGVGVA ELTEAQRRGL QVALEEFHKH
PPVQWAFQET SVESAVDTPF PAGIFVRLEF KLQQTSCRKR
DWKKPECKVR PNGRKRKCLA CIKLGSEDKV LGRLVHCPIE
TQVLREAEEH QETQCLRVQR AGEDPHSFYF PGQFAFSKAL
PRS.

Chemerin is inactive as pre-prochemerin (having SEQ ID NO: 1) and is activated through cleavage of the C-terminus and N-terminus to form a chemerin fragment having an amino acid sequence from position 21 to 157 of SEQ ID NO: 1, which can function as an agonist for CMKLR1. This chemerin fragment has the following amino acid sequence:

(SEQ ID NO: 4)
ELTEAQRRGL QVALEEFHKH PPVQWAFQET SVESAVDTPF
PAGIFVRLEF KLQQTSCRKR DWKKPECKVR PNGRKRKCLA
CIKLGSEDKV LGRLVHCPIE TQVLREAEEH QETQCLRVQR
AGEDPHSFYF PGQFAFS.

In one aspect, the present disclosure provides a composition comprising (a) chemerin or a fragment or analog thereof and (b) a lipid entity linked to the chemerin or fragment or analog thereof. Without wishing to be bound by theory, the pharmacological properties of chemerin or a fragment or analog thereof can be modulated by the choice of the lipid entity. In some embodiments, the composition of the present disclosure can function as an agonist of CMKLR1.

In some embodiments, the lipidated chemerin composition that includes a chemerin fragment consists of the sequence of YFPGQFAFS (SEQ ID NO: 2). In some embodiments, the lipidated chemerin composition that includes a chemerin fragment consists of the sequence of Y*-F-L-P-S*-Q-F-A*-Tic-S (SEQ ID NO: 3).

The chemerin analog of SEQ ID NO: 3 is found to be resistant to proteolysis. See Shimamura et al., “Identification of a stable chemerin analog with potent activity toward ChemR23,” Peptides 30, 2009, 1529-1538, the contents of which are incorporated by reference.

The opthalmic formulation of the present disclosure can be a pharmaceutical composition, which can further comprise a pharmaceutically acceptable carrier. Techniques for formulation of the disclosed compositions can be found in Remington: the Science and Practice of Pharmacy, 19^{th} edition, Mack Publishing Co., Easton, PA (1995). The pharmaceutical composition can be formulated for a variety of administration routes.

Formulations for topical administration may further comprise one or more additional ingredients.

### Methods of Treatment

The present disclosure also provides a method of treating an inflammatory condition in a subject in need thereof, the method comprising topically administering to an eye of the subject a therapeutically effective amount of the formulation disclosed herein.

The present disclosure also provides a method of treating pain in a subject in need thereof, the method comprising topically administering to an eye of the subject a therapeutically effective amount of the formulation disclosed herein.

The formulation of the present disclosure can be used to treat a variety of inflammatory conditions including, but not limited to, ocular inflammation, dry eye disease (DED), uveitis, allergic conjunctivitis, or a retinal inflammatory disease and ocular neuropathic pain.

In some embodiments, the inflammatory condition is ocular inflammation. In some embodiments, the ocular inflammation is uveitis. Uveitis is a wide range of inflammatory diseases of the eye, specifically the uvea. There are 3 basic layers of the eye—the sclera and cornea on the outside, the retina on the inside, and the uvea in between. The uvea is comprised mostly of blood vessels and connective tissue, including pigmented cells. The different parts of the uvea are the iris in the front, the ciliary body in the middle, and the choroid located behind these, which lies around most of the eye. Sometimes uveitis can affect parts of the eye other than uvea, such as retina, vitreous, or optic nerve. Types of uveitis are based on what part of the eye is affected. For example, anterior uveitis is the inflammation in the front of the eye, called iritis or iridocyclitis; intermediate uveitis is the inflammation in the middle part of the eye, or pars planitis or vitritis; posterior uveitis is the inflammation of the back of the eye, such as choroiditis, retinal vasculitis, retinitis, neuroretinitis, retinochoroiditis, or chorioretinitis.

Symptoms of uveitis commonly include redness, blurry vision, pain, light sensitivity, and floaters and flashes.

Ocular inflammation can be diagnosed through a review of illness history, slit lamp examination, blood work, or any combination thereof.

Current therapies for treating ocular inflammation include locally administered anti-cytokine or anti-inflammatory agents. In some embodiments, the formulation of the present disclosure can be administered in combination with an anti-cytokine or anti-inflammatory agent for treating ocular inflammation.

Anti-cytokine or anti-inflammatory agents include, but are not limited to, NF Kappa B inhibitors, for example corticosteroids, glucocorticoids such as flucinolonone; nonsteroidal anti-inflammatory drugs (NSAIDs) such as sulindac and tepoxalin; antioxidants such as dithiocarbamate; and other compounds such as sulfasalazine [2-hydroxy-5-[-4-[C2-pyridinylamino) sulfonyl] azo] benzoic acid], clonidine, and autologous blood-derived products such as Orthokine.

In some embodiments, the inflammatory condition is DED. DED is primarily caused by the break-down of the pre-ocular tear film which results in dehydration of the exposed outer surface. People with DED may experience irritated, gritty, scratchy or burning eyes; a feeling of something in their eyes; excess watering; and blurred vision. The definition and classification of DED can be found at “The Definition and Classification of Dry Eye Disease: Report of the Definition and Classification Subcommittee of the International Dry Eye Workshop (2007),” the Ocular Surface 2007, Vol. 5, 75-92, the contents of which are incorporated herein by reference.

DED can be diagnosed through a comprehensive eye examination. Testing, with emphasis on the evaluation of the quantity and quality of tears produced by the eyes, may include: (a) patient history to determine the patient's symptoms and to note any general health problems, medications or environmental factors that may be contributing to the dry eye problem; (b) external examination of the eye, including lid structure and blink dynamics; (c) evaluation of the eyelids and cornea using bright light and magnification; and (d) measurement of the quantity and quality of tears for any abnormalities. Special dyes may be put in the eyes to better observe tear flow and to highlight any changes to the outer surface of the eye caused by insufficient tears.

Without wishing to be bound by theory, there is a rationale that ocular inflammation as a result of pro-inflammatory cytokines and growth factors plays a major role in the underlying causes of DED. As such, locally administered anti-cytokine or anti-inflammatory agents are often used in the treatment of DED. In some embodiments, the pharmaceutical composition of the present disclosure or a composition comprising chemerin or a fragment or analog thereof can be administered in combination with an anti-cytokine or anti-inflammatory agent for treating DED.

In some embodiments, the inflammatory condition is ocular neuropathic pain. Ocular neuropathic pain can be caused by inflammation. Therefore, it can be treated by the pharmaceutical composition of the present disclosure, optionally in combination with an anti-cytokine or anti-inflammatory agent. Neuropathic pain has typical symptoms like dysesthesias (spontaneous or evoked burning pain, often with a superimposed lancinating component), but the pain may also be deep and aching. Other sensations like hyperesthesia, hyperalgesia, allodynia (pain due to a normoxious stimulus), and hyperpathia (particularly unpleasant, exaggerated pain response) may also occur.

Methods of diagnosing inflammation in the eye can be found in Teoh and Dick, “Diagnostic techniques for inflammatory eye disease: past, present and future: a review,” BMC Ophthalmology 2013, 13:41, the contents of which are incorporated herein by reference.

With respect to combination therapies involving a first therapeutic agent (e.g., a formulation of the present disclosure comprising chemerin or a fragment or analog thereof) and a second therapeutic agent (e.g., an anti-cytokine or anti-inflammatory agent), the first therapeutic agent can be administered concurrently with the second therapeutic agent; the first therapeutic agent can be administered before the second therapeutic agent; or the first therapeutic agent can be administered after the second therapeutic agent. The administrations of the first and second therapeutic agents can be separated by minutes or hours, e.g., about one hour, two hours, three hours, four hours, five hours, or six hours.

The therapeutically effective amount of a composition according to this disclosure can vary within wide limits and may be determined in a manner known in the art. For example, the composition can be dosed according to body weight. Such dosage will be adjusted to the individual requirements in each particular case including the specific compound(s) being administered, the route of administration, the condition being treated, as well as the patient being treated. In another embodiment, the drug can be administered by fixed doses, e.g., dose not adjusted according to body weight. In general, a daily dosage of from about 0.5 mg to about 1000 mg should be appropriate, although the upper limit may be exceeded when indicated. The dosage can be from about 5 mg to about 500 mg per day, e.g., about 5 mg to about 400 mg, about 5 mg to about 300 mg, about 5 mg to about 200 mg. The daily dosage can be administered as a single dose or in divided doses, or for parenteral administration it may be given as continuous infusion. The formulation of the present disclosure can be administered once a day, or several times a day, e.g., twice a day, or thrice a day. In some embodiments, the formulation of the present disclosure is administered twice a day.

In some embodiments, a therapeutically effective amount of an ophthalmic solution comprising a lipidated chimerin composition provided herein is about 0.1 mL, about 0.2 mL, about 0.3 mL, about 0.4 mL, about 0.5 mL, or about 0.6 mL. In some embodiments, a therapeutically effective amount of an ophthalmic solution as provided herein is about 0.3 mL.

The dosage regimen utilizing the formulation of the present disclosure can be selected in accordance with a variety of factors including species, ethnicity, age, weight, sex and medical condition of the patient; the severity of the condition to be treated; the route of administration; the renal and hepatic function of the patient; and the particular composition employed. An ordinarily skilled physician or veterinarian can readily determine and prescribe the effective amount of the drug required to prevent, counter, or arrest the progress of the condition.

A therapeutically effective amount of a composition is that which provides an objectively identifiable improvement as noted by the clinician or other qualified observer.

In some embodiments, a therapeutically effective amount for treating ocular inflammation is an amount that reduces the extent of inflammation in the subject by at least 10%, at least 20%, at least 30%, at least 40%, at least 50%, at least 60%, at least 70%, at least 80%, at least 90%, or at least 95% compared to a placebo.

In some embodiments, a therapeutically effective amount for treating DED is an amount that increases the production of tears in the subject by at least 10%, at least 20%, at least 30%, at least 40%, at least 50%, at least 60%, at least 70%, at least 80%, at least 90%, at least 100%, or at least 150% compared to a placebo.

In some embodiments, the efficacy of a formulation provided herein is measured according to total corneal fluorescein staining score of one or more eyes using the Ora Calibra® scale relative to the pre-treatment score.

In some embodiments, the efficacy of a formulation provided herein measured according to Ocular discomfort score of one or more eyes using the Ora Calibra® scale relative to the pre-treatment score.

The formulation disclosed herein can be included in a container, pack, or dispenser together with instructions for administration. The compositions described herein can be administered topically. For example, the composition is administered in the form of eye drops. One skilled in the art will recognize the advantages of the storage and dispensing/administering methods of formulation disclosed herein.

In some embodiments, a formulation provided herein can be included in a kit containing 1 or more ampoules. In some embodiments, a kit may contain 2, 3, 4, 5, 6, 7, 8, 9, or 10 ampoules. In some embodiments, a kit may contain about 3 ampoules. In some embodiments, each ampoule contains about 0.1 mL, about 0.2 mL, about 0.3 mL, about 0.4 mL, about 0.5 mL, or about 0.6 mL. In some embodiments, an ampoule may contain about 0.3 mL. In some embodiments a kit may contain one or more pouches, each with one or more ampoules. In some embodiments, a kit may contain one or more pouches, with two ampoules per pouch. In some embodiments, the ampoules are single use blow fill seal (BFS) ampoules. In some embodiments, the ampoules are for direct administration to the eye.

The present disclosure also provides a method of making the ophthalmic formulations described herein. In some embodiments, the method of making the ophthalmic formulations described herein comprises: (a) adding water for injection at a temperature of about 30° C. to about 80%-90% bulk batch weight of the formulation; (b) dissolving sodium phosphate monobasic monohydrate and sodium phosphate water free in the water to form a buffer solution; (c) dissolving a lipidated chemerin composition that includes a chemerin fragment consisting of the sequence of Y-F-P-G-Q-F-A-F-S (SEQ ID NO: 2) or a chemerin analog consisting of the sequence of Y*-F-L-P-S*-Q-F-A*-Tic-S (SEQ ID NO: 3), wherein * denotes D amino acids and Tic represents 1,2,3,4-tetrahydroisoquinoline-3-carboxylic acid, the chemerin fragment or chemerin analog being linked to a lipid entity via a linker;

(d) dissolving sodium chloride; (e) adjusting the temperature of the formulation to about 25° C.; (f) adjusting the pH as needed to about 7.4 to about 7.6 with 2N H₃PO₄ or 2N NaOH; (e) filtering the formulation with a sterile filter; and (f) filling a batch container aseptically. In some embodiments, the method of making produces a clear ophthalmic formulation at any of the above steps, e.g., step (b), (c), (d), and/or (f). In some embodiments, the sterile filter is a redundant 0.2 μm sterilizing grade polyethersulfone (PES) filter.

### Definitions

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein. It is to be understood that the terminology used herein is for the purpose of describing particular embodiments only, and is not intended to be limiting.

The terms “peptide,” “polypeptide,” and “protein” are used interchangeably herein and typically refer to a molecule comprising a chain of two or more amino acids (e.g., most typically L-amino acids, but also including, e.g., D-amino acids, modified amino acids, amino acid analogs, and amino acid mimetic). Peptides may be naturally occurring, synthetically produced, or recombinantly expressed. Peptides may also comprise additional groups modifying the amino acid chain, for example, functional groups added via post-translational modification. Examples of post-translation modifications include, but are not limited to, acetylation, alkylation (including methylation), biotinylation, glutamylation, glycylation, glycosylation, isoprenylation, lipoylation, phosphopantetheinylation, phosphorylation, selenation, and C-terminal amidation. The term peptide also includes peptides comprising modifications of the amino terminus and/or the carboxyl terminus. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). The term peptide also includes modifications, such as but not limited to those described above, of amino acids falling between the amino and carboxy termini. The term peptide can also include peptides modified to include one or more detectable labels.

The phrase “amino acid residue” as used herein refers to an amino acid that is incorporated into a peptide by an amide bond or an amide bond mimetic.

The terminal amino acid at one end of the peptide chain typically has a free amino group (i.e., the amino terminus or N terminus). The terminal amino acid at the other end of the chain typically has a free carboxyl group (i.e., the carboxy terminus or C terminus). Typically, the amino acids making up a peptide are numbered in order, starting at the amino terminus and increasing in the direction of the carboxy terminus of the peptide.

As used herein, the term “analog” refers to a variant or mutant polypeptide having one or more amino acid modifications compared to the wild type.

As used herein, an “amino acid modification” refers to a change in the amino acid sequence of a predetermined amino acid sequence. Exemplary modifications include an amino acid substitution, insertion and/or deletion. An “amino acid modification at” a specified position, e.g. of chemerin or a fragment thereof, refers to the substitution or deletion of the specified residue, or the insertion of at least one amino acid residue adjacent the specified residue. By insertion “adjacent” a specified residue is meant insertion within one to two residues thereof. The insertion may be N-terminal or C-terminal to the specified residue.

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (lie): leucine (Leu); lysine (Lys); methionine (Met); phenylalanine (Phe); proline (Pro); serine (Ser); threonine (Thr); tryptophan (Trp); tyrosine (Tyr); and valine (Val). Substitution with one or more non-naturally occurring amino acid residues is also encompassed by the definition of an amino acid substitution herein. A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. In some embodiments, an L amino acid can also be substituted by a D amino acid.

An “amino acid insertion” refers to the incorporation of at least one amino acid into a predetermined amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as disclosed above.

An “amino acid deletion” refers to the removal of at least one amino acid residue from a predetermined amino acid sequence.

The term “pharmaceutical composition” refers to a mixture of a compound disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers, preservatives, and adjuvants.

As used herein, the terms “treat,” “treating,” “treatment,” and the like refer to reducing or ameliorating a disorder and/or a symptom associated therewith. It will be appreciated that, although not precluded, treating a disorder or condition does not require that the disorder or symptom associated therewith be completely eliminated. The terms “treat,” “treating,” or “treatment,” do not include prevention.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

As used herein, a “subject” can be any mammal, e.g., a human, a non-human primate, mouse, rat, dog, cat, cow, horse, pig, sheep, goat, camel. In a preferred embodiment, the subject is a human.

As used herein, a “subject in need thereof” is a subject having an inflammatory condition.

As used herein, the singular forms “a,” “an” and “the” include plural referents unless the context clearly dictates otherwise. Thus, for example, reference to “a solvent” includes a combination of two or more such solvents, reference to “a peptide” includes one or more peptides, or mixtures of peptides, reference to “a drug” includes one or more drugs, reference to “a device” includes one or more devices, and the like. Unless specifically stated or obvious from context, as used herein, the term “or” is understood to be inclusive and covers both “or” and “and”.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

As used herein, the term “about,” unless indicated otherwise, refers to the recited value, e.g., amount, dose, temperature, time, percentage, etc., ±10%, ±9%, ±8%, ±7%, ±6%, ±5%, ±4%, ±3%, ±2%, or ±1%.

Unless specifically stated or obvious from context, as used herein, the term “about” when used in conjunction with numerical values and/or ranges generally refers to those numerical values and/or ranges near to a recited numerical value and/or range. In some instances, the term “about” can mean within ±10% of the recited value. For example, in some instances, “about 100 [units]” can mean within ±10% of 100 (e.g., from 90 to 110).

### EXAMPLES

The studies described herein provide the development of an optimal formulation of the lipidated chemerin product of the present disclosure (referred to as OK-101 hereafter), that exhibits long term stability and shelf-life.

### Example 1: Determining Solubility and pH Stability of OK-101 in Sodium Phosphate Buffer

### Study Design and Objective:

The solubility and pH stability of OK-101 at a target concentration of 0.05% w/v, was tested in either 50 mM or 100 mM sodium phosphate buffer. For each concentration of sodium phosphate buffer, the solubility and pH stability were determined in three separate buffer batches, each with a different starting pH.

### Results and Observations:

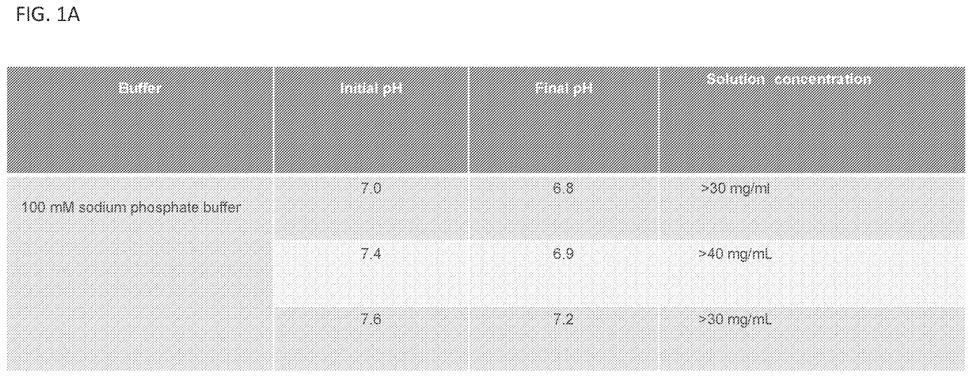

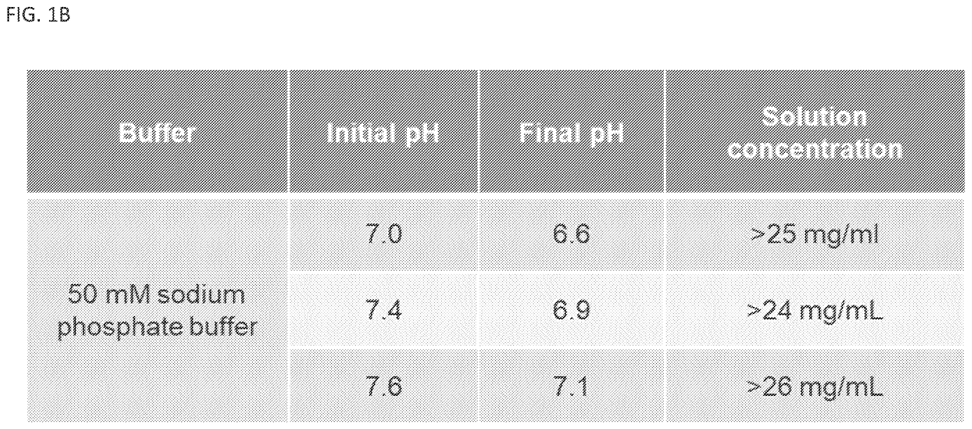

The results showed that >30 mg/ml concentration of OK-101 was observed in three batches of 100 mM sodium phosphate buffer, wherein the pH dropped slightly based on the high concentration (FIG. 1A). A concentration of >25 mg/ml of OK-101 was observed in three batches of 50 mM sodium phosphate buffer, wherein the pH dropped slightly more compared to the 100 mM sodium phosphate buffer, based on the high concentration (FIG. 1B).

### Conclusion:

The results showed that a sodium phosphate buffer concentration range of 50 mM to 100 mM is suitable develop a stable formulation of OK-101.

### Example 2: Determining Effect of Adding Osmogens on Stability and Solubility of OK-101 Formulation

### Study Design and Objective:

In the study described herein the effect of adding different osmogens (sodium chloride (NaCl) and mannitol) were tested to meet the criteria for osmolality, pH, and solubility, for generating a stable formulation of OK-101. Any adjustments needed, were done with NaOH or increasing buffer concentration.

### Results and Observations:

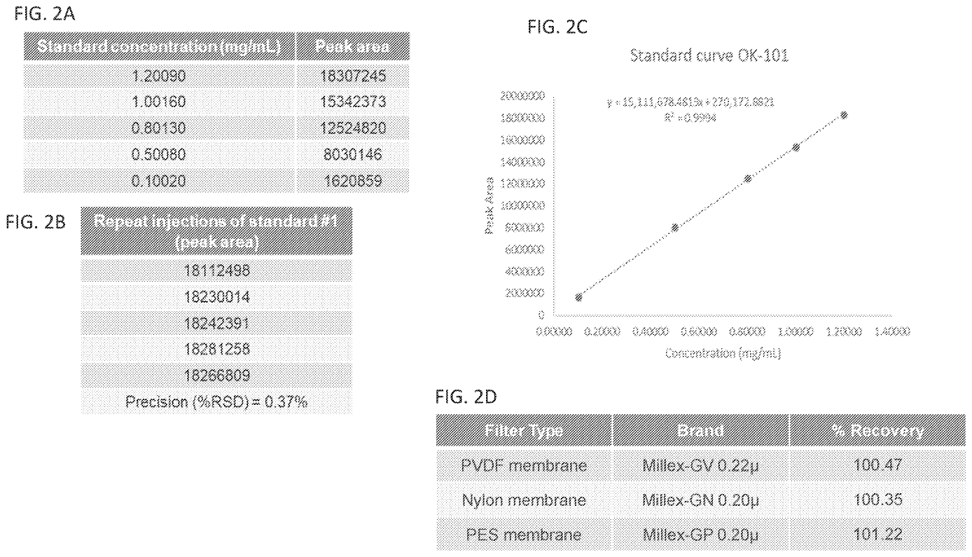

The linearity and precision of OK-101 in the buffer against a standard as determined and found to be valid, using HPLC (FIG. 2A-2C). The various 0.2-micron membrane filters that were tested for compatibility using one of the standards, for the HPLC assays us depicted in FIG. 2D. The appearance and purity criteria of the OK-101 product used for developing the formulation of the disclosure is provided in Table 1 below.

TABLE 1

| | Lot Number | | |
|---|---|---|---|
| | PD210823 | | |
| | Date of Manufacture | | |
| | 29 Oct. 2021 | | |
| Test | Specification | | Result |
| Appearance | White to | | White Powder |
| | off-white powder | | |
| Identification (MS) | 1993.1 ± 2.0 Da | | 1992.8 Da |
| Identity (HPLC) | Co-elutes with | | Conforms |
| | Reference | | |
| | standard | | |
| FTIR | Conforms | | Conforms |
| Purity (HPLC) | >93.0% (Target | | 94.6% |
| | 93.0°495.0%) | | |
| Peptide Content(% N, EA) | ≥70.0% | | 95.9% |
| Acetate Content(HPLC) | ≤10.0% | | 0.03% |
| Water Content (KF) | ≤10.0% | | 2.1% |
| Mass Balance | 95.0%-105.0% | | 98.0% |
| (% PC + % | | | |
| AcOH + % H₂O) | | | |
| Related | Report all | | RRT 0.93 0.50% |
| Substances(RP-HPLC) | 0.10°0% | | RRT 0.96 2.83% |
| | | | RRT 1.03 0.34% |
| | | | RRT 1.07 0.34% |
| | | | RRT 1.09 0.49% |
| | | | RRT 1.14 0.18% |
| | | | RRT 1.16 0.54% |
| | | | RRT 1.21 0.13% |
| Residual Organic | Report | ACN | <30 ppm |
| Solvents (GC-MS) | | IPA | <30 ppm |
| | | MTBE | <30 ppm |
| | | DMF | <30 ppm |
| | | DCM | <30 ppm |
| Endotoxin | Report | | <0.25 EU/mg |
| Bioburden | Report | TAMC | <4 CFU/100 mg |
| | | TYMC | <4 CFU/100 mg |

MS = mass spectrometry. Da = Dalton: HPLC = high performance liquid chromatography: FTIR = fourier transform infra-red spectroscopy: TBD = to be decided: EA =; AcOH - ethanol: RP-HPLC = Reversed-phase high performance liquid chromatography; RRT = relative retention time: GC-MS = gas chromatography mass spectrometry: ACN = acetonitrile: IPA = isopropyl alcohol: MTBE = Methyl tert-butyl ether: DMF = Dimethylformamide: DCM = dichloromethane: EU =- endotoxin units: TAMC = total aerobic microbial count: TYMC = total yeast/mold count.

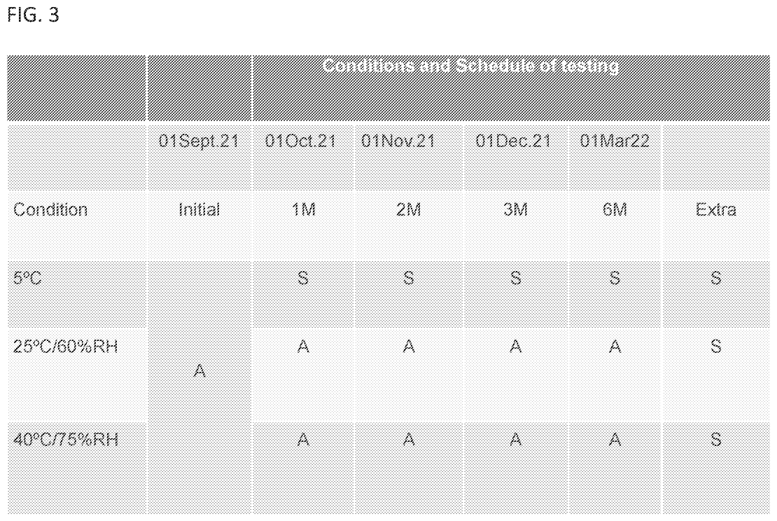

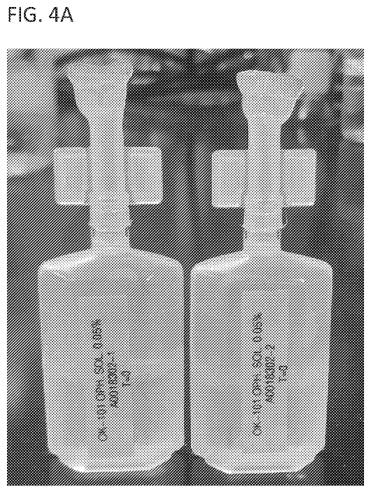

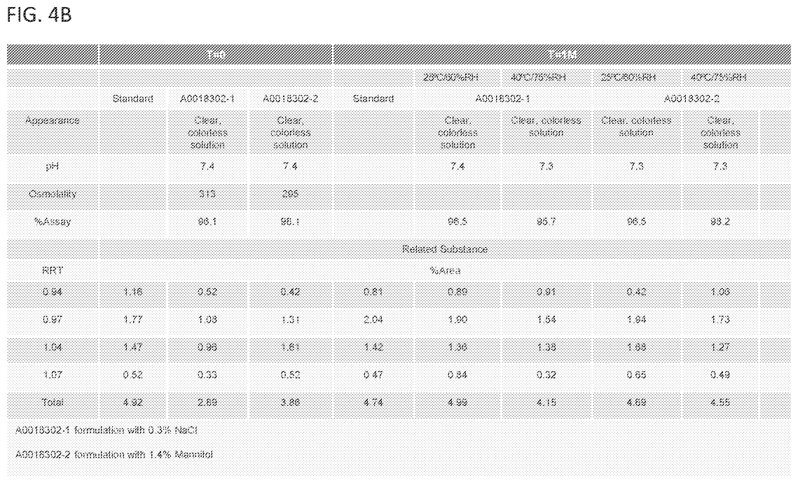

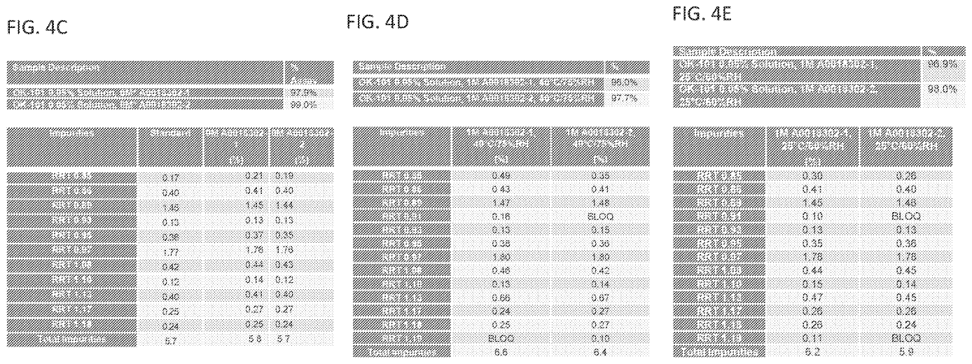

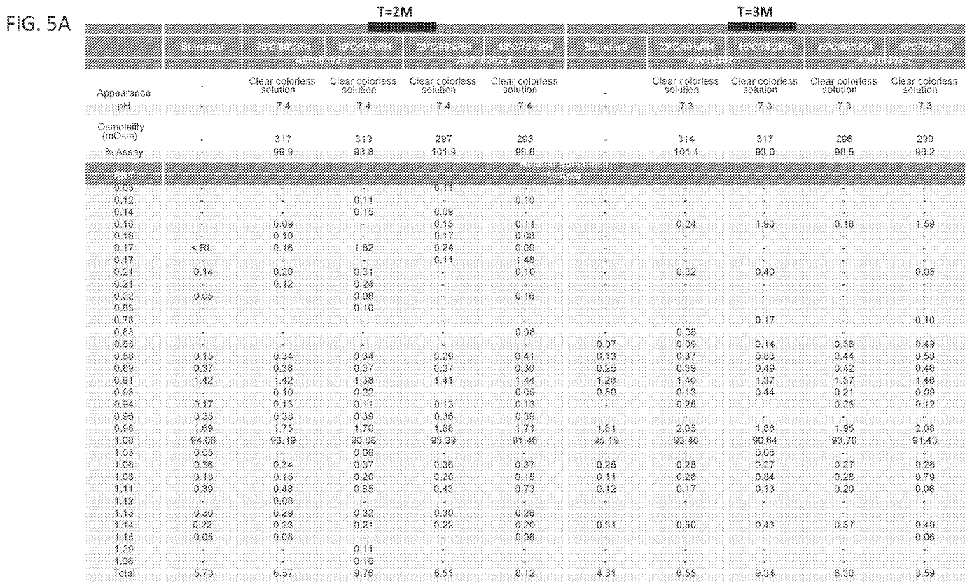

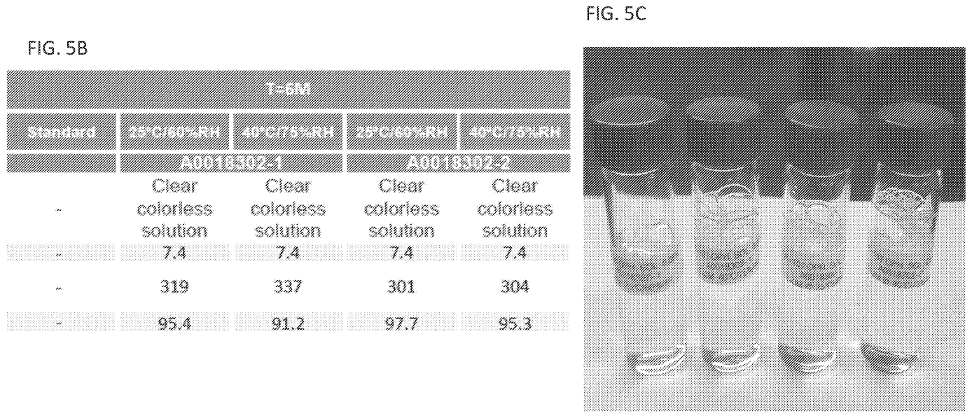

The informal stability of OK-101 in a solution with preferred osmogens was further determined based on the conditions and schedules provided in FIG. 3. The formulation was scaled up to 200 ml and filtered through a 0.22 mM PES filter, and assay results were compared. No issues were observed between unfiltered and filtered solutions. Stability was evaluated using a glass vial as one of the container closure. Two formulations of 0.05% OK-101 w/v, in 100 mM sodium phosphate buffer with two different osmogens, A0018302-1 (formulation with 0.3% NaCl) and A0018302-2 (formulation with 1.4% mannitol) were tested, as depicted in FIG. 4B. Both formulations were a clear colorless solution, as shown in FIG. 4A. The formulation was maintained over a period of one month, two month, three months, or six months, at either of the culture conditions: 25° C. and 60% relative humidity (25° C./60% RH) or 40° C. and 75% relative humidity (40° C./75% RH) (FIGS. 4B and 5A-5B. As shown in FIG. 4B, a variation of about 0.4% was observed in the activity of the OK-101 product in the formulation comprising 0.3% NaCl. Also, no significant change in pH was observed in the formulation comprising 0.3% NaCl under any of the above stated culture conditions. Observations regarding change in pH and activity, was similar in the formulation with 1.4% mannitol. A minimal variation in relative retention time, relative to the standard was observed for both the 0.3% NaCl and 1.4% mannitol formulations under both culture conditions (FIG. 4B), over a period of one month, further indicating towards the stability of the OK-101 in the formulations. Similar observations regarding relative changes in % assay purity, pH for the 1.4% mannitol formulation.

A further analysis of the both the 0.3% NaCl and 1.4% mannitol formulations with 0.05% OK-101 w/v in 100 mM sodium phosphate buffer was done under 40° C./75% RH or 25° C./60% RH, where the samples were kept at 5° C. for one month before analysis. The results showed a change in % assay purity of 1% to 1.9% depending on the culture conditions for the 0.3% NaCl formulation, as indicated in FIGS. 4C-4E. Also, there was minimal change in the RRT of the OK-101 in the 0.3% NaCl with 0.05% OK-101 w/v in 100 mM sodium phosphate buffer, stored under 40° C./75% RH or 25° C./60% RH.

### Conclusion:

The results disclosed herein showed that a formulation comprising the concentration of NaCl as disclosed herein, is stable over long periods of storage under different storage conditions, in terms of changes in pH, drug stability and purity.

### Example 3: Effect of Different Osmogens on the Change in Osmolality of the Formulations

### Study Design and Objectives:

The study described herein further studied the effect of the osmogene types and concentrations (i.e., 0.3% NaCl or 1.4% mannitol) on the stability and purity of the 0.05% OK-101 in 100 mM sodium phosphate buffer containing formulations over periods of 2, 3 and 6 months of storage period, under storage condition of 40° C./75% RH or 25° C./60% RH.

### Results and Observations:

The results described herein showed a change: a) in pH of the 0.3% NaCl formulation from 0% and 1.3% over storage periods of 2 months and 3 months, respectively. under both storage conditions. The change in osmolality was observed to be about 1.25% and 0.3% at the 25° C./60% RH storage condition at 2 months and 3 months, respectively, and 1.8% and 1.2% at the 40° C./75% RH storage condition, at 2 months and 3 months, respectively (FIG. 5A). A change in osmolality of about 1.8% at the 25° C./60% RH and 8% at the 40° C./75% RH storage conditions was observed for the period of 6 months, for the 0.3% NaCl formulation (FIG. 5B). The formulations were observed to be clear and stable, as evident from the vial pictures in FIG. 5C. Purity of the OK-101 in the formulations tested remained >90% from 2-6 months storage period under both storage conditions, with purity ranging between 95% to 98%.

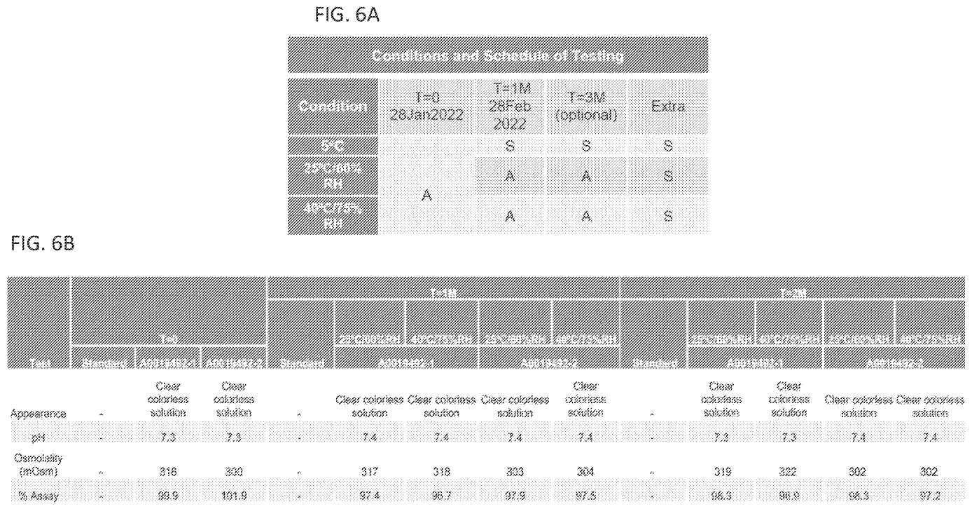

The study was further extended to determine the stability of OK-101 formulations A0019492-1 comprising 0.1% OK-101 product, in 100 mM sodium phosphate buffer with 0.3% NaCl (0.1% OK-101/0.3% NaCl formulation) or A0019492-2 comprising 0.1% OK-101 product, in 100 mM sodium phosphate buffer with 1.4% mannitol (0.1% OK-101/1.4% mannitol formulation), stored under either 25° C./60% RH or 40° C./75% RH storage conditions, based on the conditions and schedules provided in FIG. 6A The results described herein showed that the 0.1% OK-101/0.3% NaCl formulation remained colorless and clear at 1 and 2 months of storage under both the 25° C./60% RH and 40° C./75% RH storage conditions. The change in pH of the 0.1% OK-101/0.3% NaCl formulation was observed to be not more than 1.5% in pH of the formulation under at 1 and 2 months of storage both storage conditions. The change in osmolarity was observed to be about 0% and 1.2% at 1 and 2 months, respectively, under the 40° C./75% RH storage condition, and about 0.3% at both 1 and 2 months, under the 25° C./60% RH storage condition. At all time points, under both storage conditions, the purity of the 0.1% OK-101/0.3% NaCl formulation was observed to be >95%.

### Conclusion:

The results disclosed herein showed that a formulation comprising the concentration of NaCl as disclosed herein, is stable over long periods of storage under different storage conditions, in terms of changes in osmolality of the formulation.

The results of the studies described herein conclude that the lipidated chemerin composition of the present disclosure when formulated in the disclosed concentration ranges of NaCl and phosphate buffered saline (sodium phosphate buffer) and at the disclosed pH and osmolality ranges, remains stable in terms of pH and osmolality variation, appearance, and purity, for over one month to six months of storage under standard storage conditions.

### Example 4: Clinical Study Protocol Evaluating the Efficacy and Safety of an Example OK-101 Ophthalmic Solution Compared to a Placebo, in Subjects with Dry Eye Disease

The objective of this clinical study is to compare the safety and efficacy of an example OK-101 ophthalmic solution, hereinafter “OK-101 Ophthalmic Solution” to placebo for the treatment of the signs and symptoms of dry eye.

The clinical hypothesis for this study is that 0.1% OK-101 Ophthalmic Solution twice daily (BID) and 0.05% OK-101 Ophthalmic Solution BID are superior to OK-101 Placebo Ophthalmic Solution (vehicle) for the hierarchical primary endpoints of signs and symptoms of dry eye, as follows:
Sign: Total corneal fluorescein staining score of the study eye using the Ora Calibra® grading scale, measured by mean change from baseline (Visit 2, Pre-Controlled Adverse Environment [CAER]) to Visit 6.
Symptom: Ocular discomfort score of both eyes using the Ora Calibra® grading scale, measured by mean change from baseline (Visit 2, Pre-CAE®) to Visit 6.

Synopsis of Clinical study

| | |
|---|---|
| Protocol Title: | A Phase 2, Multi-center, Randomized, Double-Masked and |
| | Placebo-Controlled Study Evaluating the Efficacy and Safety of |
| | OK-101 Ophthalmic Solution Compared to Placebo in Subjects with |
| | Dry Eye |
| Study Drug: | 1) OK-101 Ophthalmic Solution 0.1% |
| | 2) OK-101 Ophthalmic Solution 0.05% |
| | 3) Placebo Ophthalmic Solution |
| Study Phase: | 2 |
| Study Objective: | The objective of this study is to compare the safety and efficacy of |
| | OK-101 Ophthalmic Solution to placebo for the treatment of the |
| | signs and symptoms of dry eye. |

Overall Study Design

| | |
|---|---|
| Structure: | Multi-center, double-masked, randomized, placebo-controlled study |
| Duration: | Approximately 14 weeks, including a 2-week run-in period and 12 |
| | weeks of treatment |
| Controls: | Placebo (vehicle minus active) Ophthalmic Solution |
| Dosage/Dose | Subjects eligible to be randomized will receive either OK-101 or |
| Regimen: | placebo to be administered bilaterally twice daily (BID) for 12 |
| | weeks (from Visit 2 to Visit 5). |
| | During a 14-day study run-in period (for the purpose of subject |
| | selection) prior to randomization, all subjects will receive Placebo |
| | Ophthalmic Solution (vehicle) bilaterally BID. |
| Summary of Visit | 6 visits over the course of approximately 9 weeks |
| Schedule: | Visit 1 = Day −14 ± 1 day, Controlled Adverse Environment |
| | (CAE) Screening |
| | Visit 2 = Day 1, CAE Confirmation/Baseline |
| | Visit 3 = Day 15 ± 2 days, 2-week CAE Challenge and Follow-Up |
| | Visit 4 = Day 29 ± 2 days, 4-week CAE Challenge and Follow-Up |
| | Visit 5 = Day 57 ± 3 days, 8-week CAE Challenge, Follow-Up |
| | Visit 6 = Day 85 ± 3 days, 12-week CAE Challenge, Follow-Up |
| | and Study Exit |
| Measures Taken to | This is a randomized treatment assignment, double- masked study. |
| Reduce Bias: | |

Study Population Characteristics

| | |
|---|---|
| Number of Subjects: | Approximately 240 (80 per treatment arm) subjects will be enrolled |
| Condition/Disease: | Dry Eye Disease |
| Inclusion Criteria: | Individuals eligible to participate in this study must meet all of the |
| | following criteria: |
| | 1. Be at least 18 years of age; |
| | 2. Provide written informed consent; |
| | 3. Be willing and able to comply with all study procedures; |
| | 4. Have a patient-reported history of dry eye for at least 6 |
| | months prior to Visit 1; |
| | 5. Have a history of use or desire to use eye drops for dry eye |
| | symptoms within 6 months of Visit 1; |
| | 6. Have a best corrected visual acuity (BCVA) of 0.7 |
| | logarithm of the minimum angle of resolution (logMAR) or |
| | better (Snellen equivalent score of 20/100 or better) in each |
| | eye at Visit 1; |
| | 7. Report a score of ≥2 according to the Ora Calibra ® Ocular |
| | Discomfort & 4-Symptom Questionnaire in at least one of |
| | the dry eye symptoms at Visits 1 and 2; |
| | 8. Have a Schirmer's Test score of ≤10 mm and ≥1 mm at |
| | Visits 1 and 2; |
| | 9. Have a corneal fluorescein staining score of ≥2 in at least |
| | one region according to the Ora Calibra ® Corneal and |
| | Conjunctival Staining Scale for Grading of Fluorescein |
| | Staining in at least one region in one eye at Visits 1 and 2 |
| | and a central score ≥1 in the same eye; |
| | 10. Have a conjunctival redness score ≥1 according to the Ora |
| | Calibra ® Conjunctival Redness for Dry Eye Scale in at least |
| | one eye at Visits 1 and 2 pre-CAE; |
| | 11. Demonstrate in the same eye(s) a response to the CAE at |
| | Visits 1 and 2 as defined by: |
| | a. Having at least a ≥1 point increase in fluorescein |
| | staining in the inferior region in at least one eye |
| | following CAE ® exposure; |
| | b. Reporting an Ocular Discomfort score ≥3 at 2 or more |
| | consecutive time points in at least one eye during |
| | CAE ® exposure (if a subject has an Ocular Discomfort |
| | rating of 3 at time = 0 for an eye, s/he must report an |
| | Ocular Discomfort rating of 4 for two consecutive |
| | measurements for that eye). Note: a subject cannot |
| | have an Ocular Discomfort score of 4 at time = 0); |
| | 12. Have at least one eye, the same eye, satisfy all criteria for 8, |
| | 9, 10 and 11 above; |
| | 13. A negative urine pregnancy test if female of childbearing |
| | potential (those who are not surgically sterilized [bilateral |
| | tubal ligation, hysterectomy or bilateral oophorectomy] or |
| | post- menopausal [12 months after last menses]) and must |
| | use adequate birth control through the study period. For |
| | non-sexually active females, abstinence may be regarded as |
| | an adequate method of birth control. |
| Exclusion Criteria: | Individuals who meet any of the following exclusion criteria will not |
| | be eligible to participate in the study: |
| | 1. Have any clinically significant slit lamp findings at Visit 1 |
| | that may include active blepharitis, meibomian gland |
| | dysfunction, lid margin inflammation, or active ocular |
| | allergies that require therapeutic treatment, and/or in the |
| | opinion of the investigator may interfere with study |
| | parameters; |
| | 2. Be diagnosed with an ongoing ocular infection (bacterial, |
| | viral, or fungal), or active ocular inflammation at Visit 1; |
| | 3. Have worn contact lenses within 7 days of Visit 1 or |
| | anticipate using contact lenses during the study; |
| | 4. Have previously had laser-assisted in situ keratomileusis |
| | (LASIK) surgery within the last 12 months; |
| | 5. Have used Restasis ®, Xiidra ®, Cequa ®, and EYSUVIS ® |
| | within 30 days of Visit 1; |
| | 6. Have had any ocular and/or lid surgeries in the past 6 |
| | months or have any planned ocular and/or lid surgeries over |
| | the study period; |
| | 7. Be using or anticipate using temporary punctal plugs during |
| | the study that have not been stable within 30 days of Visit 1; |
| | 8. Be currently taking any topical ophthalmic prescription |
| | (including medications for glaucoma) or over-the-counter |
| | solutions, artificial tears, gels or scrubs, and cannot |
| | discontinue these medications for the duration of the trial |
| | (excluding medications allowed for the conduct of the |
| | study); the respective wash-out periods are required for the |
| | following medications: |
| | a. Antihistamines (including ocular): 72 hours prior to |
| | Visit 1 |
| | b. Oral aspirin or aspirin-containing products allowed if |
| | dose has been stable over past 30 days prior to Visit 1 |
| | and no change in dose is anticipated during the study |
| | period |
| | c. Corticosteroids or mast cell stabilizers (including |
| | ocular): 14 days prior to Visit 1 Any medication (oral |
| | or topical) known to cause ocular drying that has not |
| | been administered as a stable dose for at least 30 days |
| | prior to Visit 1 and during the study |
| | d. Any medication (oral or topical) known to cause ocular |
| | drying that has not been administered as a stable dose |
| | for at least 30 days prior to Visit 1 and during the study |
| | e. All other topical ophthalmic preparations (including |
| | artificial tear substitutes) other than the study drops: 72 |
| | hours prior to Visit 1 |
| | 9. Have an uncontrolled systemic disease; |
| | 10. Be a woman who is pregnant, nursing, or planning a |
| | pregnancy; |
| | 11. Be unwilling to submit a urine pregnancy test at Visit 1 and |
| | Visit 6 (or early termination visit) if of childbearing |
| | potential. Non-childbearing potential is defined as a woman |
| | who is permanently sterilized (e.g., has had a hysterectomy |
| | or tubal ligation), or is post-menopausal (without menses |
| | for 12 consecutive months); |
| | 12. Be a woman of childbearing potential who is not using an |
| | acceptable means of birth control; acceptable methods of |
| | contraception include: hormonal - oral, implantable, |
| | injectable, or transdermal contraceptives; mechanical - |
| | spermicide in conjunction with a barrier such as a |
| | diaphragm or condom; intrauterine device; or surgical |
| | sterilization of partner. For non-sexually active females, |
| | abstinence may be regarded as an adequate method of birth |
| | control; however, if the subject becomes sexually active |
| | during the study, she must agree to use adequate birth |
| | control as defined above for the remainder of the study; |
| | 13. Have a known allergy and/or sensitivity to the test article or |
| | its components; |
| | 14. Have a condition or be in a situation which the investigator |
| | feels may put the subject at significant risk, may confound |
| | the study results, or may interfere significantly with the |
| | subject's participation in the study; |
| | 15. Have a known allergy and/or sensitivity to the test article or |
| | its components; |
| | 16. Have a condition or be in a situation which the investigator |
| | feels may put the subject at significant risk, may confound |
| | the study results, or may interfere significantly with the |
| | subject's participation in the study; |
| | 17. Be currently enrolled in an investigational drug or device |
| | study or have used an investigational drug or device within |
| | 30 days of Visit 1; |
| Study Formulations: | OK-101 Ophthalmic Solution |
| | Placebo Ophthalmic Solution |

Evaluation Criteria of Clinical Study

| | |
|---|---|
| Efficacy Measures: | Primary Efficacy Measures: |
| | The following hierarchical primary efficacy endpoints of the study |
| | are: |
| | Total corneal fluorescein staining score using the Ora |
| | Calibra ® scale, pre-CAE at Day 85 (Week 12) |
| | Ocular discomfort score using the Ora Calibra ® scale at, |
| | pre-CAE at Day 85 (Week 12) |
| | Secondary Efficacy Measures: |
| | Fluorescein staining by region: central, superior, inferior, |
| | temporal, nasal, corneal sum, conjunctival sum, and total |
| | staining using the Ora Calibra ® scale |
| | Lissamine green staining by region: central, superior, |
| | inferior, temporal, nasal, corneal sum, conjunctival sum, |
| | and total staining using the Ora Calibra ® scale |
| | Conjunctival redness using the Ora Calibra ® scale |
| | Schirmer's Test |
| | Tear film break-up time (TFBUT) |
| | Ocular Surface Disease Index © (OSDI ©) |
| | Visual Analog Score (VAS) |
| | Daily symptom diary (Ora Calibra Ocular Discomfort & |
| | 4-Symptom Questionnaire Ocular Discomfort Score) |
| | Daily symptom diary |
| | Drop comfort |
| Safety Measures: | Visual acuity |
| | Slit-lamp evaluation |
| | Adverse event query |
| | Intraocular Pressure |
| | Dilated fundoscopy |

General Statistical Methods and Types of Analyses of Clinical study
Analysis Populations

Intent-to-Treat Population - The intent-to-treat (ITT) population includes all randomized subjects. The
primary analysis will be performed on the ITT population with the Markov Chain Monte Carlo (MCMC)
imputation method for missing values. The ITT population may also be analyzed with Last Observation
Carried Forward (LOCF) imputation, imputation via pattern mixture models, and with observed data only
to assess sensitivity. Subjects in the ITT population will be analyzed as randomized.
Per Protocol Population - The per protocol (PP) population includes subjects in the ITT population who
do not have significant protocol deviations and who complete the study. Protocol deviations will be
assessed prior to database lock and unmasking. The PP population will be analyzed using observed data
only for efficacy variables. Subjects in the PP population will be analyzed as treated.
Safety Population - The safety population includes all randomized subjects who have received at least
one dose of the investigational product. The safety population will be analyzed for all safety assessments.
Subjects in the Safety population will be analyzed as treated.

Approximately 240 subjects will be randomly assigned to one of the three groups (1:1:1) to receive either OK-101 Ophthalmic Solution or placebo solution as topical ophthalmic drops administered bilaterally BID for 12 weeks. Subjects, Sponsor, Contract Research Organization (CRO), and site personnel will be masked to treatment assignment.

During the 14-day study run-in period prior to randomization, all subjects will receive OK-101 Placebo Ophthalmic Solution (vehicle) in each eye BID.

During the screening period, two 90-minute exposures to the CAE® will be conducted to ascertain eligibility to enter the study at Visit 1 (Day −14±1 day) and Visit 2 (Day 1). Subjects who qualify after the initial screening visit will enter the run-in phase, where they will self-administer vehicle BID for approximately 14 days. Those who qualify at Visit 2 (Day 1) will be randomized to receive the study drug in a double-masked fashion for 12 weeks. Subjects will self-administer drops BID and will complete daily diary assessments as instructed.

The CAE® exposure will occur at every visit with Pre-CAER, during CAE® and Post-CAE® assessments of ocular signs and symptoms.

Study drug will be discontinued at Visit 6. Subjects will exit from the study at this visit.

A study design chart is provided below:

| | |
|---|---|
| Visit 1 | Pre-CAE ® |
| (Day −14 ± 1 day): | Informed Consent/Health Insurance Portability and Accountability Act |
| CAE ® Screening | (HIPAA) |
| | Demographic Data and Medical/Medication/Ocular History |
| | Review of Inclusion/Exclusion Criteria |
| | Adverse Event Query |
| | Urine Pregnancy Testing (for females of childbearing potential) |
| | Ora Calibra Ocular Discomfort Scale |
| | Ora Calibra Ocular Discomfort and 4-symptom questionnaire |
| | Visual Analog Scale (VAS) |
| | Ocular Surface Disease Index (OSDI ©) |
| | Questionnaire |
| | Visual Acuity (Early Treatment Diabetic |
| | Retinopathy Study [ETDRS]) |
| | Slit Lamp Biomicroscopy |
| | Ora Calibra ® Conjunctival Redness Scale |
| | Ora Calibra ® Scale Fluorescein Staining |
| | Tear Film Break-up Time (TFBUT) |
| | Ora Calibra ® Scale Lissamine Green Staining |
| | Screening Challenge |
| | Subjects meeting all of the above evaluation criteria will undergo |
| | further screening evaluation in the CAE ®. Subjects will be exposed to |
| | the CAE ® for 90 minutes. Ocular Discomfort Scale (ODS) |
| | self-assessment scores will be obtained just prior to entering, during |
| | and just after the CAE ® exposure. During the CAE ® exposure, |
| | ODS scores will be collected at time 0 and every 5 minutes thereafter |
| | throughout the 90 minutes. |
| | Post-CAE ® |
| | Ora Calibra Ocular Discomfort Scale |
| | Ora Calibra Ocular Discomfort and 4-symptom questionnaire |
| | VAS |
| | Slit Lamp Biomicroscopy |
| | Ora Calibra ® Conjunctival Redness Scale |
| | Ora Calibra ® Fluorescein Staining |
| | TFBUT |
| | Ora Calibra ® Scale Lissamine Green Staining |
| | Schirmer's test |
| | At least 15-minute wait between Schirmer's test and CAE ® exposure |
| | Intraocular Pressure (IOP) |
| | Dilated Fundoscopy |
| | Review of Inclusion/Exclusion Criteria |
| | Study Drug Instillation at the Study Site |
| | Placebo Dispensation |
| | Monitoring and Query of Adverse Events (AEs) |
| | Schedule Next Visit |

Placebo Run-in Period (~14 days)

| | |
|---|---|
| Visit 2 | Pre-CAE ® |
| (Day 1): CAE ® | Study Diary/Run-in Collection |
| Confirmation/Baseline | Medical and Medication History Update |
| | Review of Inclusion/Exclusion Criteria |
| | AE query |
| | Ora Calibra Ocular Discomfort Scale |
| | Ora Calibra Ocular Discomfort and 4-symptom questionnaire |
| | VAS |
| | OSDI © Questionnaire |
| | Visual Acuity (ETDRS) |
| | Slit Lamp Biomicroscopy |
| | Ora Calibra ® Conjunctival Redness Scale |
| | TFBUT |
| | Ora Calibra ® Scale Fluorescein Staining |
| | Ora Calibra ® Scale Lissamine Green Staining |
| | Confirmatory Screening Challenge |
| | Subjects will be exposed to the CAE ® for 90 minutes. Ocular |
| | discomfort self- assessment scores (ODS) will be obtained just prior to |
| | entering, during and just after the CAE ® exposure. During the CAE ® |
| | exposure, ODS will be collected at time 0 and every 5 minutes |
| | thereafter throughout the 90 minutes. |
| | Post-CAE ® |
| | Ora Calibra Ocular Discomfort Scale |
| | Ora Calibra Ocular Discomfort and 4-symptom questionnaire |
| | VAS |
| | Slit Lamp Biomicroscopy |
| | Ora Calibra ® Conjunctival Redness Scale |
| | Ora Calibra ® Scale Fluorescein Staining |
| | TFBUT |
| | Ora Calibra ® Scale Lissamine Green Staining |
| | Schirmer's test |
| | At least 15-minute wait between Schirmer's test and CAE ® exposure |
| | Review of Inclusion/Exclusion Criteria |
| | Randomization |
| | Study Drug Instillation at the Study Site |
| | Drop Comfort Assessment |
| | Monitoring and Query of AEs |
| | Study Drug Diary/Study Drug Dispensation |
| | Subjects will be scheduled for Visit 3 |

| | |
|---|---|
| Visit 3 (Day 15 ± 2 days): | Pre-CAE ® |
| 2-week Follow-up | Study Diary/Study Drug Collection |
| Visit 4 (Day 29 ± 2 days): | Medical |
| 4-week Follow-up | Medication History Update |
| Visit 5 (Day 57 ± 3 days): | Review of Inclusion/Exclusion Criteria |
| 8-week Follow-up | AE query |
| Visit 6 (Day 85 ± 3 days): | Ocular Discomfort as measured by VAS |
| 12-week Follow-up | OSDI © Questionnaire |
| and Study Exit | Ora Calibra Ocular Discomfort Scale |
| | Ora Calibra Ocular Discomfort and 4-symptom questionnaire |
| | VAS |
| | Visual Acuity (ETDRS) |
| | Ora Calibra ® Conjunctival Redness Scale |
| | Slit Lamp Biomicroscopy |
| | Ora Calibra ® Scale Fluorescein Staining |
| | TFBUT |
| | Ora Calibra ® Scale Lissamine Green Staining |
| | 90-minute CAE exposure |
| | Ora Calibra ® Ocular Discomfort Scale upon entering the CAE and |
| | every 5 minutes thereafter |
| | CAE ® exposure: Subjects will be exposed to the CAE ® for 90 minutes. |
| | Ocular discomfort self- assessment scores (ODS) will be obtained just |
| | prior to entering, during and just after the CAE ® exposure. During the |
| | CAE ® exposure, ODS will be collected at time 0 and every 5 minutes |
| | thereafter throughout the 90 minutes. |
| | Post-CAE ® |
| | Ora Calibra Ocular Discomfort Scale |
| | Ora Calibra Ocular Discomfort and 4-symptom questionnaire |
| | VAS |
| | Slit Lamp Biomicroscopy |
| | Ora Calibra ® Conjunctival Redness Scale |
| | Ora Calibra ® Scale Fluorescein Staining |
| | TFBUT |
| | Ora Calibra ® Scale Lissamine Green Staining |
| | Schirmer's test |
| | At least 15-minute wait between Schirmer's test and CAE ® exposure |
| | Review of Inclusion/Exclusion Criteria |
| | Randomization |
| | Study Drug Instillation at the Study Site |
| | Drop Comfort Assessment |
| | Monitoring and Query of AEs |
| | Study Drug Diary/Study Drug Dispensation |
| | Subjects will be scheduled for next visit or Study Exit (Visit 6) |

### Efficacy Endpoints

For efficacy endpoints, the unit of analysis will be the study eye, or the “worst eye,” as defined by the following:

Worst Eye: Eyes are eligible for analysis if they meet all of the inclusion criteria. At least one eye (the right eye or the left eye) must meet all of the criteria. In the case that both eyes are eligible for analysis, the eye with the worst total corneal fluorescein staining score (Ora Calibra® scale) at pre-CAE® baseline will be selected as the study eye. If both eyes have the same total corneal fluorescein staining score at baseline, the right eye (OD) will be selected as the study eye.

### Safety Measures

The safety measures being evaluated are: Visual acuity, Slit-lamp evaluation, Adverse event query, Intraocular Pressure, and Dilated fundoscopy.

### Study Treatments

OK-101 Ophthalmic Solution will be formulated as a sterile solution at pH 6.5 for topical ophthalmic administration and is intended for clinical use. The study drug will be supplied in blow-fill seal ampoules, which allow for product administration directly to the eye. Each ampoule will contain a nominal volume of 0.3 mL.

The excipients which will be used to manufacture OK-101 Ophthalmic Solution will be standard excipients for use in ophthalmic solutions that comply with their respective United States Pharmacopeia (USP)/European Pharmacopoeia (EP) monographs.

The placebo for OK-101 Ophthalmic Solution contains all the same excipients used in the active formulation without the peptide.

### Description of Route of Administration, Dosage, Dosage Regimen, and Treatment Period

The dosage and dosage regimen were selected based on positive efficacy results in the proof-of-concept nonclinical studies. The proposed treatment period is 12 weeks.

### Instructions for Use and Administration

Subjects will receive OK-101 Placebo Ophthalmic Solution (vehicle) at Visit 1, and assigned study drug kit at Visits 2, 3, 4, and 5.

Subjects who are randomized must administer study drug in each eye BID. At Visit 2, subjects will self-administer one dose of study drug in office.

### Labeling/Packaging

Investigational product (IP) will be packaged and labeled into clinical kits. The primary packaging of the OK-101 Ophthalmic Solution will be blow-fill-seal ampules with a fill volume of 0.3 mL. The secondary packaging is a foil pouch that contains three ampules in each pouch.

### Screening Run-in Period

For the run-in screening period, 12 pouches of 3 ampoules will be packaged in a 2-week clinical kit. Each subject will receive 1 kit.

### Treatment Period

BID Dosing: For the treatment period, 12 pouches of 3 ampoules will be packaged in a 2-week clinical kit. Each subject will receive 6 kits total, with each subject receiving 1 kit per visit on Visit 2 (Day 1) and Visit 3 (Day 15) and 2 kits per visit at Visit 4 (Visit 29) and Visit 5 (Day 57).

### Treatment Groups

Subjects will be stratified by the following signs and symptom:

Total corneal fluorescein staining score of the study eye (Ora Calibra® scale) at Visit 2, pre-CAE (<=5; >5).

Ocular discomfort score using the Ora Calibra® scale at Visit 2, pre-CAE (<=2; >2).

### Study Duration

An individual subject's participation will involve 6 visits over approximately a 99-day period (14 days pre-screening, 85 days of treatment).

### Example 5: Description of Manufacturing Process and Process Controls of an Example OK-101 Ophthalmic Solution

As described in Table 2, the example OK-101 ophthalmic solution, hereinafter “OK-101 Ophthalmic Solution,” is manufactured aseptically via sterile filtration utilizing redundant 0.2 μm sterilizing grade polyethersulfone (PES) filters. The solution has a pH of 7.4-7.6, which is suitable for topical ophthalmic administration.

OK-101 Ophthalmic Solution is filled/primary packaged in single use blow-fill seal (BFS) ampoules, which enable the product to be administered directly to the eye. Each ampoule contains a nominal volume of 0.3 mL. The secondary packaging is a foil pouch that contains two ampoules in each pouch. The development batch finished drug product was stored at 25±2° C./60±5% relative humidity (RH). Based on available stability data the clinical batch finished drug product is stored at 2-8° C.

The manufacturing process and controls for OK-101 Ophthalmic Solution are performed in compliance with current Good Manufacturing Practices. The manufacture of each active drug concentration is similar except the amount of drug substance added. The manufacture of placebo drug product is similar to that of both active drug product concentrations except the drug substance is omitted.

TABLE 2
OK-101 Ophthalmic Solution Manufacturing Process Flow

| | | IN-PROCESS |
|---|---|---|
| STEP | DESCRIPTION | CONTROL |
| Step 1 | Addition of target of 30° C. of WFI to 85% bulk | Not Applicable |
| | batch weight | |
| Step 2 | Dissolve sodium phosphate monobasic monohydrate, and | Appearance: complete |
| | sodium phosphate water free | dissolution |
| Step 3 | Dissolve OK-101 Peptide in buffer solution | Appearance: complete |
| | | dissolution |
| Step 4 | Dissolve Sodium Chloride | Appearance: complete |
| | | dissolution |
| Step 5 | Adjust temperature to 25 C. | Not Applicable |
| Step 6 | Check pH and adjust, if necessary, with 2N H₃PO₄ | Appearance: complete |
| | or 2N NaOH. | dissolution |
| | | pH: 7.4-7.6 |
| Step 7 | q.s. to final batch size | Weight check |
| | | pH: 7.4-7.6 |
| Step 8 | Bioburden Reduction Filtration | Post-use Filter Integrity |
| | | Test |
| Step 9 | Sterile Filtration | Bioburden, Pre- and Post- |
| | | use Filter Integrity Tests |
| Step 10 | Aseptic Filling | 100% Container Closure |
| | | Integrity Testing, Fill |
| | | Volume, 100% inspection, |
| | | AQL |
| Step 11 | OK-101 Ophthalmic Solution | |

AQL = Acceptable Quality Limit; HCl = Hydrochloric acid; NaOH = Sodium hydroxide; qs = quantum sufficit or quantitate sufficiently; WFI = water for injection.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents, patent applications and references mentioned throughout the specification of the present invention are herein incorporated in their entirety by reference.

The invention embraces all combinations of preferred and more preferred groups and suitable and more suitable groups and embodiments of groups recited above.

What is claimed is:

1. An ophthalmic formulation comprising:
(a) about 0.1% to about 0.5% w/v of NaCl;
(b) about 25 mM to about 100 mM of phosphate buffered saline; and
(c) about 0.05% to about 0.1% w/v of a lipidated chemerin composition that includes a chemerin fragment consisting of the sequence of Y-F-P-G-Q-F-A-F-S (SEQ ID NO: 2) or a chemerin analog consisting of the sequence of Y*-F-L-P-S*-Q-F-A*-Tic-S (SEQ ID NO: 3), wherein * denotes D amino acids and Tic represents 1,2,3,4-tetrahydroisoquinoline-3-carboxylic acid, the chemerin fragment or chemerin analog being linked to a lipid entity via a linker;
wherein the formulation has a pH of about 6.5 to about 8.5, and an osmolality of about 200 to about 450 mOsm/Kg.

2. The formulation of claim 1, wherein the pH of the formulation changes by no more than 1.5% over a period of one to six months when stored at 25° C. to 40° C. and 60% to 75% relative humidity.

3. The formulation of claim 1 or 2, wherein the formulation comprises NaCl at a concentration of about 0.1% to about 0.3% w/v.

4. The formulation of claim 3, wherein the formulation comprises NaCl at a concentration of about 0.3% w/v.

5. The formulation of any one of the proceeding claims, wherein the formulation comprises phosphate buffered saline at a concentration of about 25 mM to about 50 mM.

6. The formulation of claim 5, wherein the formulation comprises phosphate buffered saline at a concentration of about 50 mM.

7. The formulation of any one of the proceeding claims, wherein the formulation comprises the lipidated chemerin composition at a concentration of about 0.05% w/v.

8. The formulation of any one of claims 1-6, wherein the formulation comprises the lipidated chemerin composition at a concentration of about 0.1% w/v.

9. The formulation of any one of the proceeding claims, wherein the formulation has a pH of about 7.4 to about 7.6.

10. The formulation of any one of the proceeding claims, wherein the formulation has a pH of about 7.2 to about 7.4.

11. The formulation of any one of the proceeding claims, wherein the formulation has an osmolality of about 313 to about 322 mOsm/kg.

12. The formulation of claim 11, wherein the formulation has an osmolality of about 314 to about 319 mOsm/Kg

13. The formulation of any one of the proceeding claims, wherein the pH of the formulation changes by between 1% and 1.5% over a period of six months, when stored at 25° C. and 60% relative humidity.

14. The formulation of any one of the proceeding claims, wherein the pH of the formulation changes by between 1% and 1.5% over a period of six months, when stored at 40° C. and 75% relative humidity.

15. The formulation of any one of claims 1-12, wherein the pH of the formulation does not change over a period of six months, when stored at 25° C. and 60% relative humidity.

16. The formulation of any one of claims 1-12, wherein the pH of the formulation does not change over a period of six months, when stored at 40° C. and 75% relative humidity.

17. The formulation of any one of claims 1-12, wherein the osmolality of the formulation changes by no more than 10% over a period of one to six months when stored at 25° C. to 40° C. and 60% to 75% relative humidity.

18. The formulation of any one of claims 1-13 and 15, wherein the osmolality of the formulation changes by between 0.25% to 2% over a period of six months, when stored at 25° C. and 60% relative humidity.

19. The formulation of any one of claims 1-12, 14 and 16-17, wherein the osmolality of the formulation changes by between 1% and 8% over a period of six months, when stored at 40° C. and 75% relative humidity.

20. The formulation of any one of claims 1-13 and 15, wherein the osmolality of the formulation does not change over a period of six months, when stored at 25° C. and 60% relative humidity.

21. The formulation of any one of claims 1-12, 14 and 16-17, wherein the osmolality of the formulation does not change over a period of six months, when stored at 40° C. and 75% relative humidity.

22. The formulation of any one of claims 1-21, wherein the purity of the lipidated chemerin composition used for making the formulation determined by HPLC is ≥94.6% and peptide content is ≥95.9

23. The formulation of claim 22, wherein the purity of the lipidated chemerin composition used for making the formulation is 95% to 98%.

24. The formulation of any one claims 1-23, wherein Tic represents

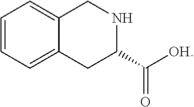

25. The formulation of any one of claims 1-24, wherein the linker is selected from the group consisting of:

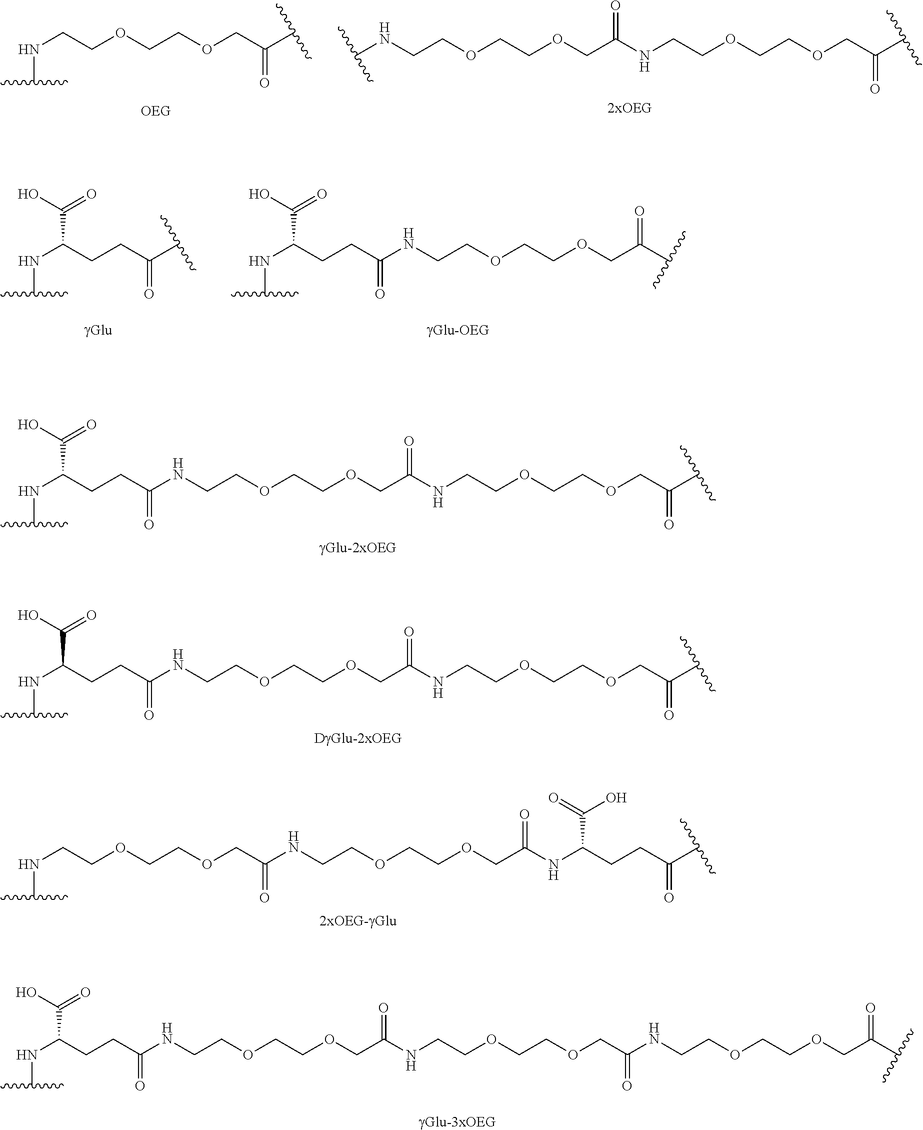

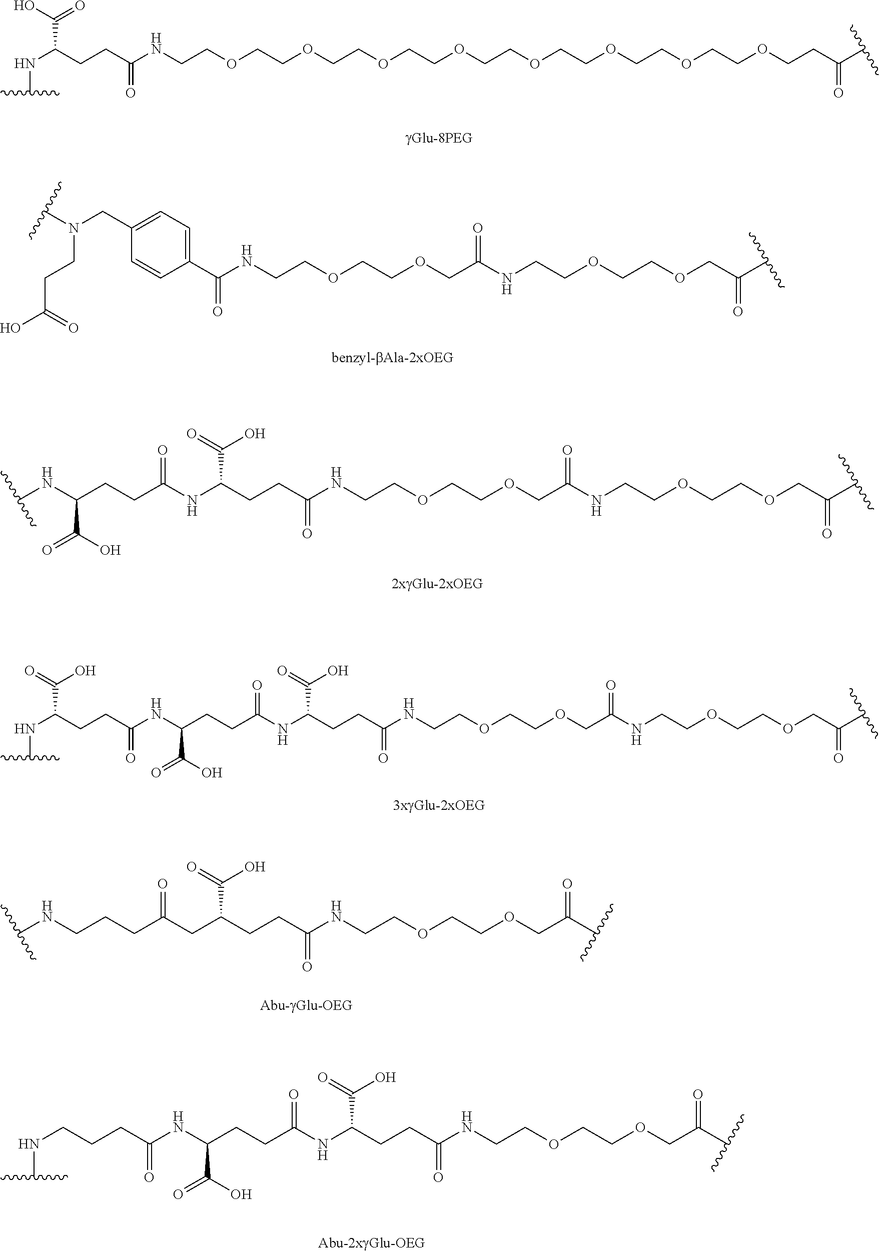 [SMILES: *N[C@@H](CCC(=O)NCCOCCOCCOCCOCCOCCOCCOCCOCCC(*)=O)C(=O)O ; *C(=O)COCCOCCNC(=O)COCCOCCNC(=O)c1ccc(CN(*)CCC(=O)O)cc1 ; *N[C@@H](CCC(=O)N[C@@H](CCC(=O)NCCOCCOCC(=O)NCCOCCOCC(*)=O)C(=O)O)C(=O)O ; *N[C@@H](CCC(=O)N[C@@H](CCC(=O)N[C@@H](CCC(=O)NCCOCCOCC(=O)NCCOCCOCC(*)=O)C(=O)O)C(=O)O)C(=O)O ; *NCCCC(=O)C[C@@H](CCC(=O)NCCOCCOCC(*)=O)C(=O)O ; *NCCCC(=O)N[C@@H](CCC(=O)N[C@@H](CCC(=O)NCCOCCOCC(*)=O)C(=O)O)C(=O)O]

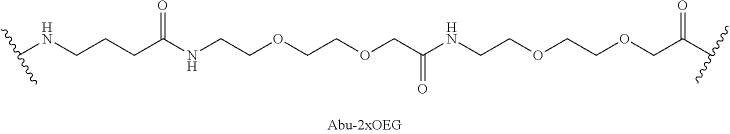 [SMILES: *NCCCC(=O)NCCOCCOCC(=O)NCCOCCOCC(*)=O]

26. The formulation of any one of claims 1-24, wherein the linker comprises polyethylene glycol, GG, KGG, or a combination thereof.

27. The formulation of any one of claims 1-26, wherein the lipid entity is linked at or near the N-terminus of the chemerin fragment or chemerin analog.

28. The formulation of any one of claims 1-26, wherein the lipid entity is linked at or near the C-terminus of the chemerin fragment or chemerin analog.

29. The formulation of claim 1, wherein the lipidated chemerin composition has the following structure:

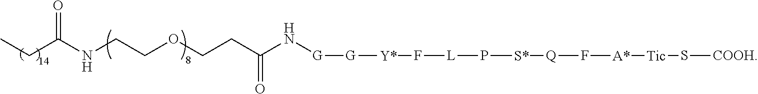

30. The formulation of any one of claims 1-29, formulated for topical administration as eye drops.

31. A method of treating an inflammatory condition in a subject in need thereof, the method comprising topically administering to an eye of the subject a therapeutically effective amount of the formulation of any one of claims 1-30.

32. The method of claim 30, wherein the inflammatory condition is dry eye disease, uveitis, allergic conjunctivitis, or a retinal inflammatory disease.

33. A method of treating pain in a subject in need thereof, the method comprising topically administering to an eye of the subject a therapeutically effective amount of the formulation of any one of claims 1-30.

34. The method of any one of claims 31 to 33, wherein the formulation is administered once a day, twice a day, or thrice a day.

35. The method of any one of claims 31 to 34, wherein the subject is human.

36. The method of claim 35, wherein the formulation is administered twice daily.

37. The method of any one of claims 31 to 36, wherein the efficacy of the treatment is measured by a total corneal fluorescein staining and/or ocular discomfort relative score relative to a pre-treatment score.

38. A kit for administration to a subject in need thereof, wherein the kit comprises one or more ampoules, and wherein the ampoules contain about 0.3 mL of the formulation of claim 30, and instructions for use.

39. The kit of claim 38, comprising one or more foil pouches, wherein each pouch contains two ampoules.

40. The kit of claim 38 or claim 39, wherein the ampoules are single use blow-fill seal (BFS) ampoules.

41. A method of making the formulation of claim 30 comprising:
(a) adding water for injection at a temperature of about 30° C. to about 80%-90% bulk batch weight of the formulation;
(b) dissolving sodium phosphate monobasic monohydrate and sodium phosphate water free in the water to form a buffer solution;
(c) dissolving a lipidated chemerin composition that includes a chemerin fragment consisting of the sequence of Y-F-P-G-Q-F-A-F-S (SEQ ID NO: 2) or a chemerin analog consisting of the sequence of Y*-F-L-P-S*-Q-F-A*-Tic-S (SEQ ID NO: 3), wherein * denotes D amino acids and Tic represents 1,2,3,4-tetrahydroisoquinoline-3-carboxylic acid, the chemerin fragment or chemerin analog being linked to a lipid entity via a linker;
(d) dissolving sodium chloride;
(e) adjusting the temperature of the formulation to about 25° C.;
(f) adjusting the pH as needed to about 7.4 to about 7.6 with 2N H₃PO₄ or 2N NaOH;
(e) filtering the formulation with a sterile filter; and
(f) filling a batch container aseptically.

42. The method of making of claim 41, wherein any one of steps (b), (c), (d), or (f) produce a clear formulation.

43. The method of making of claim 41 or claim 42, wherein the sterile filter is a redundant 0.2 μm sterilizing grade polyethersulfone (PES) filter.
